(19) United States
(12) Patent Application Publication
(10) Pub. No.: US 2012/0179184 A1
(43) Pub. Date: Jul. 12, 2012

(54) HEART VALVE REMODELING

(76) Inventor: Boris Orlov, Haifa (IL)

(21) Appl. No.: 13/496,226

(22) PCT Filed: Sep. 15, 2010
(86) PCT No.: PCT/IL10/00756

§ 371 Date: Mar. 15, 2012

Related U.S. Application Data
(60) Provisional application No. 61/242,403, filed on Sep. 15, 2009.

(51) Int. Cl. A61B 17/32

(52) U.S. Cl. 606/170

(57) ABSTRACT

A device for remodeling atrioventricular valve leaflets including one or more stretching members and one or more leaflet engaging elements, wherein each stretching member includes at least one leaflet engaging element, the leaflet engaging elements are configured to penetrate a valve leaflet, and the stretching members are configured to be positioned externally to a plane of the leaflet when the leaflet engaging elements have penetrated a valve leaflet. A method for remodeling atrioventricular valve leaflets including inserting one or more leaflet engaging elements into an atrioventricular valve leaflet, and using stretching members positioned externally to a plane of the leaflet to apply a stretching force on the atrioventricular valve leaflet. Related apparatus and methods are also described.

### FIELD AND BACKGROUND OF THE INVENTION

The present invention, in some embodiments thereof, relates to the field of cardiac medicine and more particularly but not exclusively to atrioventricular valve leaflet remodeling.

An aspect of some embodiments of the invention relates to improving the functioning of the mitral valve or the tricuspid valve. In the following, provided is a brief explanation of the structure of the mitral valve and its relation to the functioning of the heart in general. Afterwards, a brief explanation of mitral valve insufficiency and some treatments of this condition are described.

The Human Heart and the Mitral Valve

Reference is now made to FIG. 1, which is a prior art schematic depiction of a healthy heart in cross section.

The human heart 10, depicted in cross sectional long axis view in FIG. 1, is a muscular organ that pumps deoxygenated blood through the lungs to oxygenate the blood and pumps oxygenated blood to the rest of the body by rhythmic contractions of four chambers.

After having circulated in the body, deoxygenated blood from the body enters the right atrium 12 through the vena cava 14. Right atrium 12 contracts, pumping the blood through a tricuspid valve 16 into the right ventricle 18. Right ventricle 18 contracts, pumping the blood through the pulmonary semi-lunar valve 20 into the pulmonary artery 22 which splits to two branches, one for each lung. The blood is oxygenated while passing through the lungs and reenters the heart to the left atrium 24.

Left atrium 24 contracts, pumping the oxygenated blood through the mitral valve 26 into the left ventricle 28. Left ventricle 28 contracts, pumping the oxygenated blood through the aortic semi-lunar valve 30 into the aorta 32. From aorta 32, the oxygenated blood is distributed to the rest of the body.

Reference is now made to FIGS. 2A and 2B, which are schematic prior art depictions of parts of a healthy heart.

Physically separating left ventricle 28 and right ventricle 18 is interventricular septum 33. Physically separating left atrium 24 and right atrium 12 is an interatrial septum 25 (FIG. 2B).

Mitral valve 26, depicted in FIG. 2A (top view) and in FIG. 2B (cross sectional long axis view) is defined by an approximately circular mitral annulus 34 that defines a mitral orifice 36. Attached to the periphery of mitral annulus 34 is an anterior leaflet 38 and a smaller posterior leaflet 40, the anterior leaflet 38 and the posterior leaflet 40 joined at commissures 41. Each leaflet is between about 0.8 and 2.4 mm thick and composed of three layers of soft tissue. The typical area of mitral orifice 36 in a healthy adult is between 4 and 6 cm2 while the typical total surface area of the anterior leaflet 38 and the posterior leaflet 40 is approximately 12 cm2. Consequently and as depicted in FIG. 2B, the anterior leaflet 38 and the posterior leaflet 40 curve downwards into left ventricle 28 and coapt to accommodate the excess leaflet surface area, producing a coaptation depth 42 that constitutes a seal. The typical length of coaptation depth 42 in a healthy heart 10 of an adult is approximately 7-8 mm.

The bottom surface of the anterior leaflet 38 and the posterior leaflet 40 are connected to papillary muscles 44 at the bottom of left ventricle 28 by posterior chordae 46 and anterior chordae 48.

During diastole, left atrium 24 contracts to pump blood downwards into left ventricle 28 through mitral valve 26. The blood flows through mitral orifice 36 pushing the anterior leaflet 38 and the posterior leaflet 40 downwards into left ventricle 28 with little resistance.

During systole left ventricle 28 contracts to pump blood upwards into aorta 32 through aortic semi-lunar valve 30. Mitral annulus 34 contracts pushing the anterior leaflet 38 and the posterior leaflet 40 inwards and downwards, reducing the area of mitral orifice 36 by about 20% to 30% and increasing the length of coaptation depth 42. The pressure of blood in left ventricle 28 pushes against the bottom surfaces of the anterior leaflet 38 and the posterior leaflet 40, tightly pressing the anterior leaflet 38 and the posterior leaflet 40 together at coaptation depth 42 so that a tight leak-proof seal is formed. To prevent prolapse of the anterior leaflet 38 and the posterior leaflet 40 upwards into left atrium 24, papillary muscles 44 contract pulling the edges of the anterior leaflet 38 and the posterior leaflet 40 downwards through posterior chordae 46 and anterior chordae 48, respectively.

Mitral Valve Insufficiency

An effective seal of mitral valve 26 is dependent on a sufficient degree of coaptation, in terms of length, area and continuity of coaptation depth 42. If coaptation depth 42 is insufficient or non-existent, there is mitral valve insufficiency, that is, regurgitation of blood from left ventricle 28 up into left atrium 24. A lack of sufficient coaptation may be caused by any number of physical anomalies that allow leaflet prolapse (for example, elongated or ruptured chordae 46 and 48, weak papillary muscles 44) or prevent coaptation (for example, short chordae 46 and 48, the anterior leaflet 38 and the posterior leaflet 40).

Mitral valve insufficiency leads to many complications including arrhythmia, atrial fibrillation, cardiac palpitations, chest pain, congestive heart failure, fainting, fatigue, low cardiac output, orthopnea, paroxysmal nocturnal dyspnea, pulmonary edema, shortness of breath, and sudden death.

There are a number of pathologies that lead to a mitral valve insufficiency including collagen vascular disease, ischemic mitral regurgitation, myxomatous degeneration of the anterior leaflet 38 and the posterior leaflet 40 and rheumatic heart disease

In ischemic mitral regurgitation (resulting, for example, from myocardial infarction, chronic heart failure), the anterior leaflet 38 and the posterior leaflet 40 and chordae 46 and 48 have normal structure and the mitral valve insufficiency results from altered geometry of left ventricle 28. It is believed that as a result of ischemia, portions of the heart walls necrose. During healing, the necrotic tissue is replaced with unorganized tissue leading to remodeling of the heart which reduces coaptation through distortion of mitral annulus 34 and sagging of the outer wall of left ventricle 28 which displaces papillary muscles 44.

Reference is now made to FIGS. 2C and 2D, which are schematic depictions of (prior art) parts of a heart with mild ischemic mitral regurgitation related to incomplete coaptation of the leaflets of the mitral valve.

In FIGS. 2C (top view) and 2D (cross sectional long axis view), The reduction of coaptation resulting from ischemia is depicted for a mitral valve 26 of an ischemic heart 50 that has undergone mild remodeling and suffers from ischemic mitral regurgitation. In FIG. 2D is seen how an outer wall of left ventricle 28 sags outwards, displacing papillary muscles 44 downwards which, through chordae 46 and 48, pulls the anterior leaflet 38 and the posterior leaflet 40 downwards and apart, reducing coaptation. The incomplete closure of mitral valve 26 is seen in FIGS. 2C and 2D.

In some cases, the following progression is observed. Initially, ischemic mitral regurgitation is a minor problem, typically leading only to shortness of breath during physical exercise due to the fact that a small fraction of blood pumped by left ventricle 28 is pumped into left atrium 24 and not through aortic semi-lunar valve 30, reducing heart capacity. To compensate for the reduced capacity, left ventricle 28 beats harder and consequently remodeling continues. Ultimately leaflet coaptation is entirely eliminated as the anterior leaflet 38 and the posterior leaflet 40 are pulled further and further apart, leading to more blood regurgitation, further increasing the load on left ventricle 28, and further remodeling. Ultimately, the left side of the heart fails and the person dies.

Apart from humans, mammals that suffer from mitral valve insufficiency include horses, cats, dogs, cows and pigs.

Currently, it is accepted to use open-heart surgical methods to improve mitral valve functioning by many different methods including: modifying the subvalvular apparatus (for example, lengthening the chordae) to improve coaptation; by implanting an annuloplasty ring, (for example, as described in U.S. Pat. Nos. 3,656,185, 6,183,512 and 6,250,308) to force mitral valve annulus 34 into a normal shape; or by implanting devices in the mitral valve to act as prosthetic leaflets (for example, the United States Patent applications published as US 2002/065554, US 2003/0033009, US 2004/0138745 or US 2005/0038509).

Surgical augmentation of a mitral valve anterior leaflet 38 for improving mitral valve leaflet coaptation for treating ischemic mitral valve regurgitation is taught by Kincaid E H, Riley R D, Hines M H, Hammon J W and Kon N D in Ann. Thorac. Surg. 2004, 78, 564-568. First, an incision is made in the anterior leaflet almost from commissure to commissure. The edges of a roughly elliptical patch of material (for example, bovine pericardium, 1 cm wide by 3 cm long) are sutured to either side of the incision augmenting the anterior leaflet by an amount roughly equal to the surface area of the patch. Additionally, a flexible annuloplasty ring is implanted to reshape the mitral annulus. Although possibly effective, such augmentation is considered a complex surgical procedure performed only by cardiac surgeons having above average skill.

Open heart surgery of any kind is complex, requires long recovery time and is accompanied by a high rate of complications and death. The failure rate of operations for mitral valve function improvement is unacceptably high. Even when successfully performed, persons having undergone such surgeries have an increased chance of infection and stroke, and are often required to use anticoagulant agents for the rest of their lives. Often there is a need to repeat the surgery after a few years.

For these reasons, such procedures are usually tried only when the degree of mitral valve insufficiency is such that death is likely or imminent.

Following a myocardial infarction, the one-year mortality of persons with no ischemic mitral regurgitation is about 6%, with mild ischemic mitral regurgitation about 10%, with moderate ischemic mitral regurgitation about 17% and with severe ischemic mitral regurgitation approximately 40%.

Background art includes:
U.S. Pat. No. 3,656,185 to Carpentier;
U.S. Pat. No. 6,183,512 to Howanec, Jr. et al;
U.S. Pat. No. 6,250,308 to Cox;
Published US patent application number 2002/065554 of Streeter;
Published US patent application number 2003/0033009 of Gabbay;
Published US patent application number 2004/0138745 of Macoviak;
Published US patent application number 2005/0038509 of Ashe;
PCT published patent application WO2008/149355 of Mor Research Applications Ltd., herein incorporated in its entirety by reference into the specification;
PCT patent application IL2008/001565 of Mor Research Applications Ltd. herein incorporated in its entirety by reference into the specification;
an article by Kincaid E H, Riley R D, Hines M H, Hammon J W, and Kon N D, in Ann. Thorac. Surg. 2004, 78, 564-568; and
an article titled “Mitral Leaflet Adaptation to Ventricular Remodeling:

Occurrence and Adequacy in Patients With Functional Mitral Regurgitation” by Chaput M., Handschumacher M., Tournoux, F., Hua L., Guerrero L. L., Vlahakes G. J., Levine, R. A.' in Circulation—Journal of the American Heart Association, published online 4 Aug. 2008.

### SUMMARY OF THE INVENTION

The present invention, in some embodiments thereof, relates to the field of cardiac medicine and more particularly but not exclusively to atrioventricular valve leaflet remodeling.

The present invention, in some embodiments thereof, includes a device which attaches to one or more locations of an atrioventricular valve leaflet, and stretches the leaflet, thereby remodeling, that is reshaping, the leaflet. The remodeled leaflet optionally provides improved coaptation in the atrioventricular valve.

In some embodiments, the device consists essentially of bio-compatible material, that is, material suitable for implantation in a body

In some embodiments the remodeling is optionally achieved by forcing apart the leaflet locations to which the device is attached, permanently stretching the leaflet.

In some embodiments the remodeling is optionally achieved by forcing apart the leaflet locations to which the device is attached beyond an elastic limit of the leaflet, thereby permanently, plastically deforming the leaflet even if the device is later detached from the leaflet.

In some embodiments the remodeling is optionally achieved by forcing apart the leaflet locations to which the device is attached at less than an elastic limit of the leaflet.

A first stage of stretching optionally includes a stretching soon after first embedding the device in a leaflet. The first stage optionally includes an elastic stretching, and an optional plastic deformation—stretching beyond elasticity. A second stage of stretching optionally includes a further stretching while the device is embedded in the leaflet, by an adaptation made by the leaflet.

In some embodiments of the invention the device stretches the leaflet using elastic, spring-like members to exert a stretching force. In some embodiments of the invention the device stretches the leaflet when a surgeon manipulates the device such that the device forcefully expands after attaching to the leaflet.

In some embodiments of the invention the device stretches the leaflet by engaging two or more locations on the leaflet and exerting a stretching force between the two or more locations.

In some embodiments of the invention the device stretches the leaflet by engaging one or more locations on the leaflet, by anchoring to a location outside the valve leaflet, such as an annulus, and exerting a stretching force between the anchoring location and the one or more locations on the leaflet.

In some embodiments of the invention the device stretches the leaflet by engaging one or more locations on the leaflet, by anchoring to a location outside the valve leaflet, such as a ventricle wall or an interventricular septum, and exerting a pulling force between the anchoring location and the one or more locations on the leaflet.

In some embodiments of the invention the device stretches a leaflet which has been slit in one or more locations. The slit and/or slits enable stretching and/or assist controlling a shape of a stretched leaflet.

In some embodiments of the invention the device is biodegradable, and exits a heart after performing the stretching, whether by pulling and/or by pushing.

One or more leaflets in atrioventricular valves may be remodeled in a procedure which includes use of embodiments of the invention.

Leaflets in other valves and/or other parts of the body may also be remodeled using embodiments of the invention. A non-limiting example includes the tricuspid valve.

The present invention, in some embodiments thereof, is optionally delivered via a catheter, preferably in an operation on a beating heart.

The present invention, in some embodiments thereof, optionally does not cut into or engage a leaflet's hinge.

The present invention, in some embodiments thereof, optionally does not cut into or engage a leaflet's lips, which perform the coaptation.

According to an aspect of some embodiments of the present invention there is provided a device for remodeling atrioventricular valve leaflets including one or more stretching members and one or more leaflet engaging elements, wherein each stretching member includes at least one leaflet engaging element, the leaflet engaging elements are configured to penetrate a valve leaflet, and the stretching members are configured to be positioned externally to a plane of the leaflet when the leaflet engaging elements have penetrated a valve leaflet.

According to some embodiments of the invention, the stretching members are configured to exert a stretching force on the valve leaflet by pushing on the leaflet engaging elements.

According to some embodiments of the invention, the stretching members are configured to exert a stretching force on the valve leaflet by pulling on the leaflet engaging elements.

According to some embodiments of the invention, the stretching members and the leaflet engaging elements are constructed of biodegradable material.

According to some embodiments of the invention, the leaflet engaging element includes a sharp tip for penetrating the valve leaflet and a backward directed hook for hindering the leaflet engaging element from detaching from the valve leaflet.

According to some embodiments of the invention, the leaflet engaging element includes a blade.

According to some embodiments of the invention, the leaflet engaging elements are configured to engage the valve leaflet substantially in a shape which is one of a group including a circle, an oval, a closed curve, two parallel lines, and a plurality of concentric circles.

According to some embodiments of the invention, further including an anchor configured to attach to an anchoring location in a heart.

According to some embodiments of the invention, the anchoring location is one of a group including a coronary sinus, a mitral annulus, an interatrial septum, an interventricular septum, and a ventricle wall.

According to some embodiments of the invention, the leaflet engaging elements are configured to penetrate the leaflet over an area including about 20% to 80% of an area of the leaflet.

According to some embodiments of the invention, the leaflet engaging elements are configured to penetrate the leaflet over an area including a diameter of about 12 mm.

According to some embodiments of the invention, the device is further configured for opening so that the leaflet engaging elements stretch the area to a diameter including about 20 mm.

According to some embodiments of the invention, the device is further configured to be flexible so as to be capable of folding into a flexible catheter.

According to some embodiments of the invention, the device is further configured to deploy leaflet engaging elements over a leaflet area including a diameter of about 12 mm.

According to some embodiments of the invention, at least one of the stretching members includes a spring.

According to some embodiments of the invention, the device including at least five stretching members.

According to some embodiments of the invention, at least one of the stretching members is attached to a first connecting member, and at least another stretching member is attached to a second connecting member, and the first connecting member and the second connecting member are connected by a spring.

According to some embodiments of the invention, the device further including a mesh including a shape configured for covering at least holes made by the leaflet engaging elements.

According to some embodiments of the invention, the device further including a counter plate, the counter plate including blades for cutting into the valve leaflet, the blades configured to cut into the valve leaflet within an area encompassed by the stretching members. According to some embodiments of the invention, the counter plate is further configured to be flexible so as to be capable of folding into a catheter.

According to some embodiments of the invention, at least one of the one or more stretching members is connected to a stem, and the counter plate further includes a hole for sliding over the stem.

According to some embodiments of the invention, the device further including a mesh for covering at least holes made by the blades.

According to an aspect of some embodiments of the present invention there is provided a method for remodeling atrioventricular valve leaflets including inserting one or more leaflet engaging elements into an atrioventricular valve leaflet, and using stretching members positioned externally to a plane of the leaflet to apply a stretching force on the atrioventricular valve leaflet.

According to some embodiments of the invention, the stretching force is applied by forcing apart the leaflet engaging elements. According to some embodiments of the invention, the stretching force is applied by pushing on the leaflet engaging elements. According to some embodiments of the invention, the stretching force is applied by pulling on the leaflet engaging elements.

According to some embodiments of the invention, the stretching force is enough to cause elastic stretching of the leaflet, and less than enough to cause plastic deformation of the leaflet.

According to some embodiments of the invention, the stretching force is enough to cause plastic deformation of the leaflet.

According to some embodiments of the invention, the leaflet engaging elements are left in the atrioventricular valve leaflet and the forcing continues over a period of time.

According to some embodiments of the invention, the leaflet engaging elements are implanted in the atrioventricular valve leaflet.

According to some embodiments of the invention, further including covering, by a mesh, holes made by the leaflet engaging elements.

According to some embodiments of the invention, further including using stretching fingers attached to the leaflet engaging elements for forcing apart the leaflet engaging elements.

According to some embodiments of the invention, further including using connecting members attached to the leaflet engaging elements, and using one or more springs attached to the connecting members for forcing apart the leaflet engaging elements.

According to some embodiments of the invention, the method further including cutting holes into the atrioventricular valve leaflet.

According to some embodiments of the invention, further including covering holes made by the cutting with a mesh.

According to some embodiments of the invention, the method further including bringing the one or more leaflet engaging elements up to an in-vivo atrioventricular valve leaflet via a catheter.

According to some embodiments of the invention, further including, for a mitral valve leaflet, performing the bringing via one of the following group of approaches transapically, via an apex of a left ventricle, via an aorta and a pulmonary semi-lunar valve, via a coronary sinus to a left atrium, via an interventricular septum, and via an interatrial septum.

According to an aspect of some embodiments of the present invention there is provided a device for remodeling atrioventricular valve leaflets including one or more means for grasping one or more locations on an atrioventricular valve leaflet, and means, positioned externally to a plane of the leaflet, for applying a stretching force between the locations.

According to some embodiments of the invention, further including means for cutting holes into the atrioventricular valve leaflet.

According to some embodiments of the invention, further including means for covering up the holes in the atrioventricular valve leaflet.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic (prior art) depiction of a healthy heart in cross section;

FIGS. 2A and 2B are schematic (prior art) depictions of parts of a healthy heart;

FIGS. 2C and 2D are schematic (prior art) depictions of parts of a heart with mild ischemic mitral regurgitation related to incomplete coaptation of the leaflets of the mitral valve;

FIG. 3A is a simplified flowchart of a method of operation of embodiments of the invention;

FIG. 3B is a more detailed flowchart of the method of operation of FIG. 3A;

FIG. 4A is a simplified view of a first example embodiment of the invention, just prior to engaging a leaflet;

FIG. 4B is a simplified view of the embodiment of FIG. 4A, after engaging the leaflet;

FIG. 5A is a simplified view of a second leaflet stretching device constructed according to another example embodiment of the invention, having different length stretching fingers, just prior to engaging a leaflet;

FIG. 5B is a simplified view of the leaflet stretching device of FIG. 5A, after engaging the leaflet;

FIG. 6A is a simplified view of a third leaflet stretching device constructed according to yet another example embodiment of the invention, having equal length stretching fingers and having guide wires attached to each finger;

FIG. 6B is a simplified view of the third leaflet stretching device of FIG. 6A;

FIG. 6C is a simplified view of the third leaflet stretching device of FIG. 6A, drawn in context of an atrioventricular valve;

FIG. 6D is a simplified view of the third leaflet stretching device of FIG. 6A, further having an anchor for anchoring in a heart, drawn in context of an example approach to the mitral valve;

FIG. 6E is a simplified view of the third leaflet stretching device of FIG. 6A, drawn in context of an alternative example approach to the mitral valve;

FIG. 6F is a simplified view of the third leaflet stretching device of FIG. 6A, depicting a stretching force of the stretching fingers, controlled in part by a lip of the catheter;

FIG. 6G is a simplified view of the third leaflet stretching device of FIG. 6A, attached by the stretching fingers to the anterior leaflet of the mitral valve;

FIG. 7A is a simplified view of a fourth leaflet stretching device, constructed according to yet another example embodiment of the invention;

FIG. 7B is a simplified view of the fourth leaflet stretching device, in an unfolded state;

FIG. 7C is a simplified view of the fourth leaflet stretching device, embedded in an example atrioventricular leaflet;

FIG. 8 is a simplified view of a fifth leaflet stretching device, constructed according to yet another example embodiment of the invention;

FIG. 9 is a simplified view of a sixth leaflet stretching device, constructed according to yet another example embodiment of the invention;

FIG. 10 is a simplified view of a seventh leaflet stretching device, constructed according to yet another example embodiment of the invention;

FIGS. 11A and 11B are simplified views of a leaflet stretching device anchored through a left atrium wall to the coronary sinus;

FIGS. 12A and 12B are simplified views of an eighth leaflet stretching device, constructed according to yet another example embodiment of the invention;

FIG. 13 is a simplified image of a mesh for optional use with a leaflet stretching device which cuts holes through a leaflet;

FIGS. 14A, 14B, and 14C are simplified views of the eighth leaflet stretching device of FIG. 12A partially open and embedded into an atrioventricular valve leaflet;

FIGS. 15A, 15B, and 15C are simplified views of the eighth leaflet stretching device of FIG. 12A fully open and embedded into an atrioventricular valve leaflet;

FIG. 16 is a simplified view of the eighth leaflet stretching device of FIG. 12A fully open and embedded into an atrioventricular valve leaflet, and an optional mesh closing upon an opposite side of the atrioventricular valve leaflet;

FIGS. 17A to 17H are simplified views a ninth leaflet stretching device, constructed according to yet another example embodiment of the invention, and operation of the ninth leaflet stretching device in context of an atrioventricular valve leaflet;

FIG. 18 is a simplified view of a tenth leaflet stretching device, constructed according to yet another example embodiment of the invention, in context of an atrioventricular valve leaflet;

FIGS. 19A to 19D are simplified views of a bottom, transapical, approach to the mitral valve and an example embodiment of a leaflet stretching device;

FIG. 20 is a simplified view of another bottom approach to a mitral valve;

FIG. 21 is a simplified view of combined top and bottom approaches to a mitral valve 26;

FIGS. 22A-22E are simplified drawings of example prongs constructed according to the present invention; and

FIGS. 23A-23E are simplified drawings of example embodiments of the present invention, designed for leaflet stretching working on one side of a leaflet, penetrating through the leaflet, and working on two sides of the leaflet.

### DESCRIPTION OF SPECIFIC EMBODIMENTS OF THE INVENTION

The present invention, in some embodiments thereof, relates to the field of cardiac medicine and more particularly but not exclusively to atrioventricular valve leaflet remodeling.

The present invention, in some embodiments thereof, includes a device which attaches to several locations of an atrioventricular valve leaflet, and stretches the leaflet, thereby remodeling, that is reshaping, the leaflet. The remodeled leaflet provides improved coaptation in the atrioventricular valve.

In some embodiments the remodeling is optionally achieved by forcing apart the leaflet locations to which the device is attached, permanently stretching the leaflet. In some embodiments the remodeling is optionally achieved by forcing apart the leaflet locations to which the device is attached beyond an elastic limit of the leaflet, thereby permanently deforming the leaflet even if the device is later detached from the leaflet.

In some embodiments of the invention the device stretches the leaflet using elastic, spring-like members to exert a stretching force. In some embodiments of the invention the device stretches the leaflet when a surgeon manipulates the device such that the device forcefully expands after attaching to the leaflet.

One or more leaflets in atrioventricular valves may be remodeled in a procedure which includes use of embodiments of the invention. Leaflets in other valves and/or other parts of the body may also be remodeled using embodiments of the invention.

The present invention, in some embodiments thereof, is optionally delivered via a catheter, preferably in an operation on a beating heart.

The present invention, in some embodiments thereof, is optionally delivered via open heart surgery.

The present invention, in some embodiments thereof, applies particularly to the mitral valve and the tricuspid valve.

Leaflet material has a property that when stretched, and especially if also cut, the leaflet remodels. Some information about the property may be learned from an article titled “Mitral Leaflet Adaptation to Ventricular Remodeling: Occurrence and Adequacy in Patients With Functional Mitral Regurgitation” by Chaput M., Handschumacher M., Tournoux, F., Hua L., Guerrero L. L., Vlahakes G. J., Levine, R. A.' in Circulation—Journal of the American Heart Association, published online 4 Aug. 2008, which is hereby incorporated by reference in its entirety into the specification.

Apparently even a force which is naturally present and exerted on the leaflet is enough to stretch a leaflet. A larger force than which is naturally present is even more able to cause stretching.

The present invention, in some embodiments thereof, stretches one or more leaflets of an atrioventricular valve, causing deformation of the one or more leaflets. The deformation of the leaflet remodels the leaflet, optionally improving coaptation of the valve leaflets.

In some embodiments of the invention, a central portion of the leaflet is the portion which is most substantially stretched.

Descriptions which are provided herein with reference to a specific one of the atrioventricular valves refer to any one of the atrioventricular valves, and descriptions which are provided herein with reference to a specific one leaflet of the atrioventricular valves refer to any one of the leaflets of the Descriptions which are provided herein with reference to a specific one of the atrioventricular valves.

Embodiments of the invention include both methods for inserting deforming apparatuses into a heart, and the deforming apparatuses.

The present invention, in some embodiments thereof, includes, by way of a non-limiting example, the following methods of using catheters to approach the mitral valve: a top approach from the left atrium; a bottom approach from the left ventricle; and a combined approach, using catheters from both top and bottom. Regardless of whether the valve is approached by a top approach or a bottom approach, either top or bottom of the leaflet may be contacted and attached for stretching.

The present invention, in some embodiments thereof, includes, by way of a non-limiting example, the following approaches to the mitral valve:

a top, inter-atrial approach;

a top approach, via the left atrium;

a top approach, via the coronary sinus into the left atrium, similar to an approach described in PCT patent application IL2008/001565 of Mor Research Applications Ltd;

a bottom approach, via the aorta and the aortic semi-lunar valve to the left ventricle, also termed a trans-aortic approach;

a bottom, transapical, approach; and

a bottom approach, via the interventicular septum.

The present invention, in some embodiments thereof, includes, by way of a non-limiting example, the following methods of using catheters to approach the tricuspid valve: a top approach from the right atrium; a bottom approach from the right ventricle; and a combined approach, using catheters from both top and bottom.

The present invention, in some embodiments thereof, includes, by way of a non-limiting example, the following approaches to the tricuspid valve:

a top approach, via the vena cava and the right atrium; and

a bottom approach, via the interventicular septum.

Descriptions of the invention are typically provided with reference to the mitral valve or the tricuspid valve. What is described with reference to the mitral valve is applicable to the tricuspid valve, and vice versa, with a caveat that top, bottom, and combined approaches to one valve should be changed, as appropriate, to top, bottom, and combined approaches to the other valve.

Descriptions of the invention apply to leaflets in other valves, which may also be remodeled using embodiments of the invention.

By way of a non-limiting example, one approach to the tricuspid valve is an anterior approach.

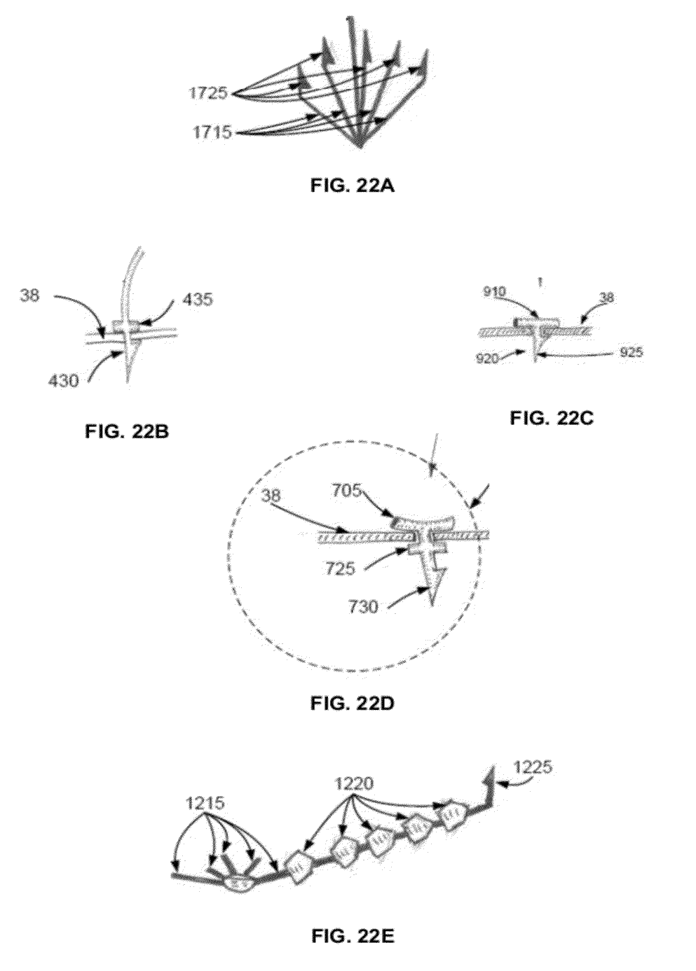

The present invention, in some embodiments thereof, includes prongs, or pins, which penetrate at least some of the leaflet, in order to exert sideways pressure and stretch the leaflet. The prongs may include different shapes, such as, by way of a non-limiting example: as pins with an outward curve, pointing in a stretching direction, designed to prevent slipping out of the leaflet, having a suitable angle between the prongs and a leaflet surface and between the prongs and stretching arms of the stretching device; as pins having a hook at their tip to prevent the tip slipping out of the leaflet; as blades which both cut into the leaflet, part and/or all the way through, and stretch the leaflet perpendicular to the blade; and as pins intended to penetrate through the leaflet and mate with a corresponding cavity in apparatus placed on an opposite side from the pin entrance. Example embodiments of the prongs are described below, with reference to exemplary embodiments of the invention, and with reference to FIGS. 22A-22E.

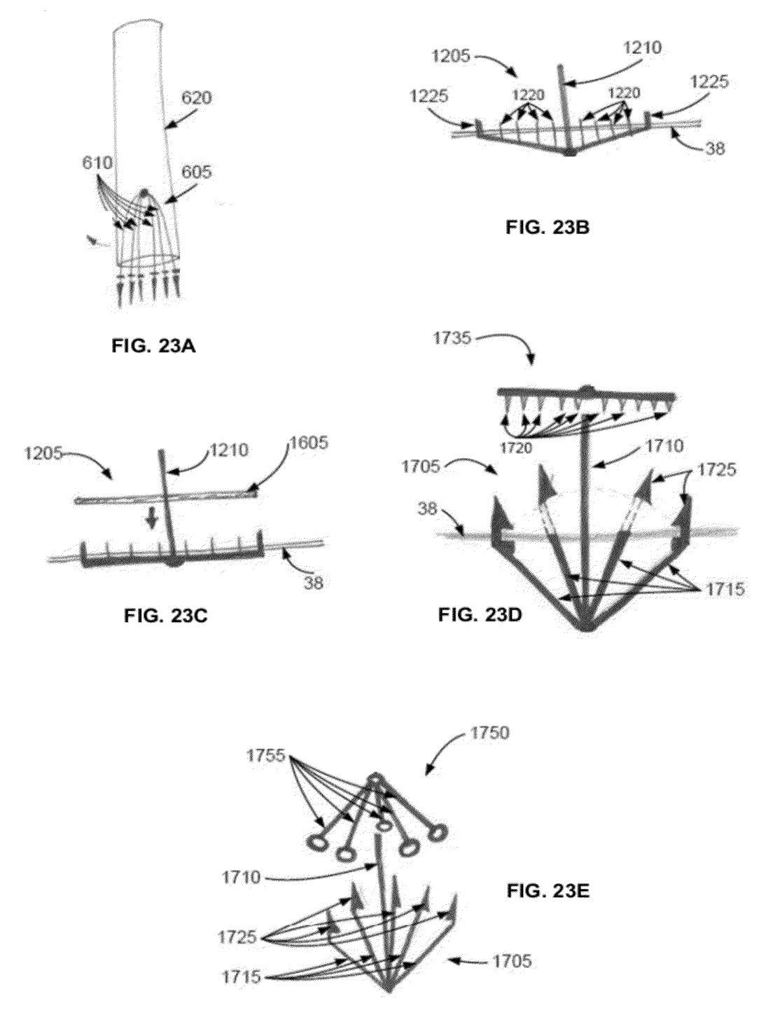

The present invention, in some embodiments thereof, includes a first stretching device on one side of a leaflet, and an optional second device on the other side of the leaflet. The optional second device may also include pins of some type for stretching, and/or mating cavities for accepting pins of the first device. Example embodiments of a leaflet stretching device working on one side of a leaflet, penetrating the leaflet, and working on both sides of the leaflet are described below, with reference to exemplary embodiments of the invention, and with reference to FIGS. 23A-23E.

The present invention, in some embodiments thereof, optionally includes a mesh placed on the leaflet surface. The mesh optionally seals holes in the leaflet if a penetrating hole was cut through the leaflet. The mesh optionally helps remodel the leaflet for improved coaptation.

The present invention, in some embodiments thereof, is designed to remain within a body. Alternative embodiments of the invention are designed to leave some or all parts within a body for as long as stretching is desired, typically until a deformation becomes permanent, and later be retrieved and extracted from the body and/or be made of materials which decompose in a body over a time longer than the desired stretching time.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

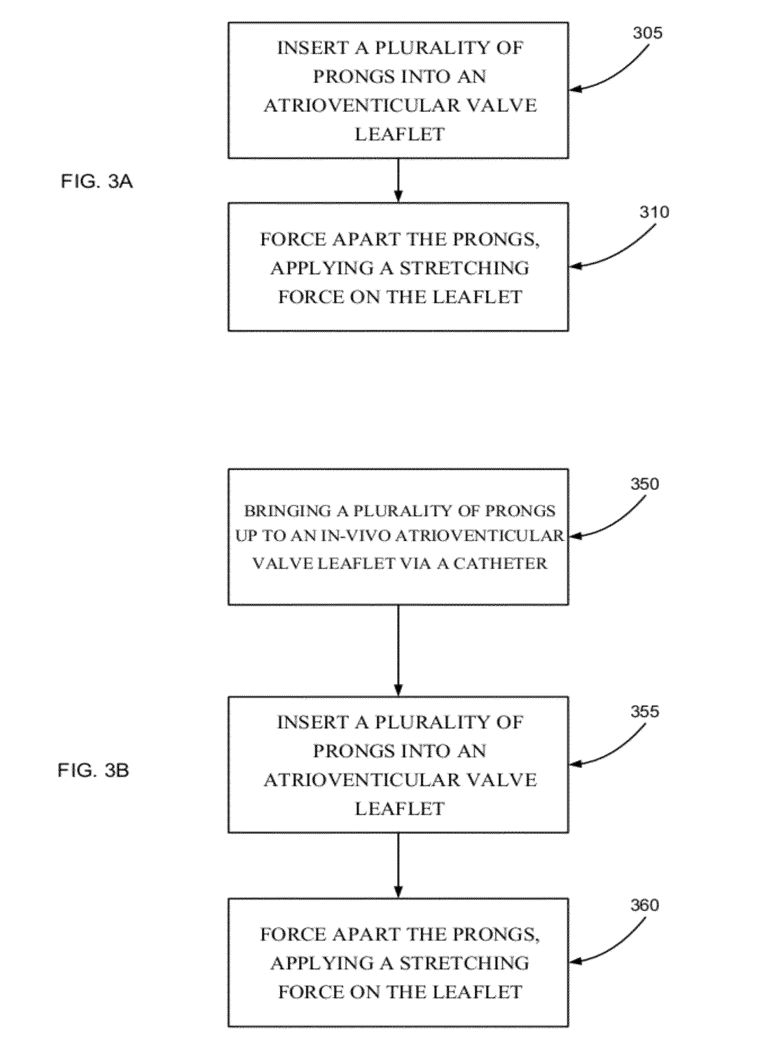

Reference is now made to FIG. 3A, which is a simplified flowchart of a method of operation of embodiments of the invention.

In order to remodel an atrioventricular valve leaflet, a plurality of prongs is inserted into an atrioventricular valve leaflet (305).

Once the prongs engage the atrioventricular valve leaflet, the prongs are forced apart, applying a stretching force on the leaflet (310).

The leaflet stretches somewhat upon application of the force. The application of a stretching force may continue over time, continuing the stretching.

The present invention includes methods of bring the prongs described in FIG. 3A up to an in-vivo atrioventricular valve leaflet, even up to a beating heart atrioventricular valve leaflet.

Reference is now additionally made to FIG. 3B, which is a more detailed flowchart of the method of operation of FIG. 3A.

A plurality of prongs is brought up to an in-vivo atrioventricular valve leaflet via a catheter (350). The plurality of prongs is inserted into an atrioventricular valve leaflet (355), and the prongs are forced apart, applying a stretching force on the leaflet (360).

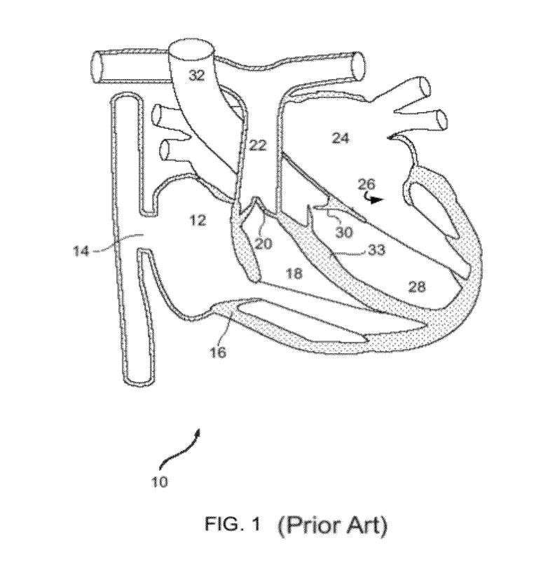

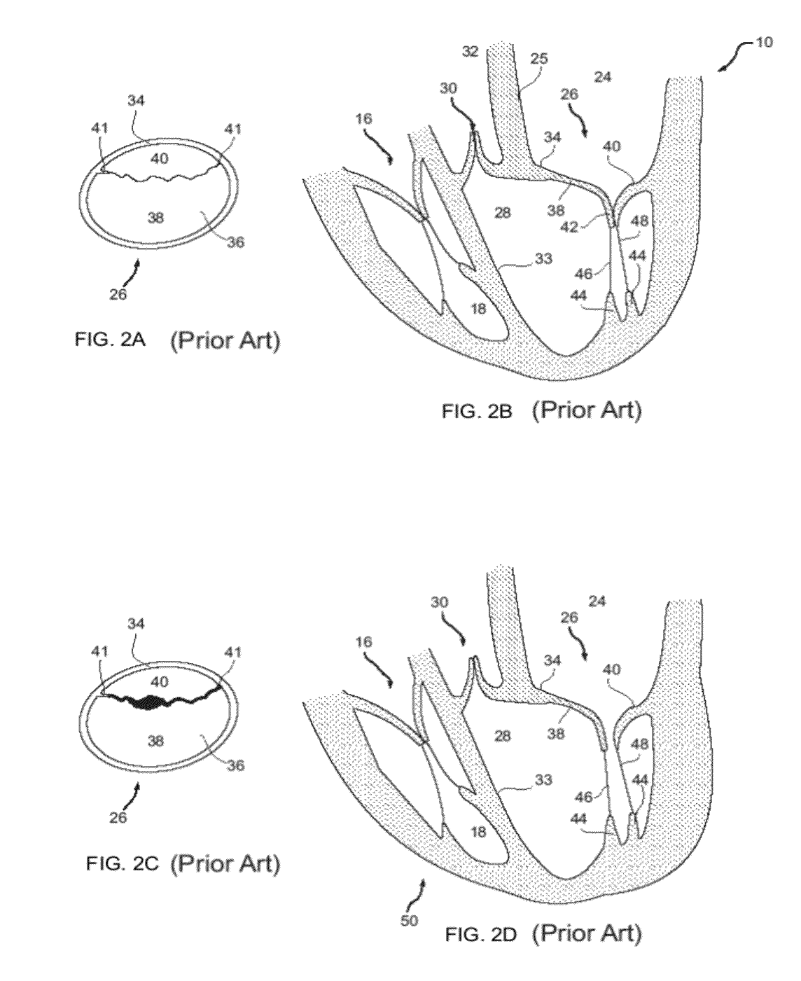

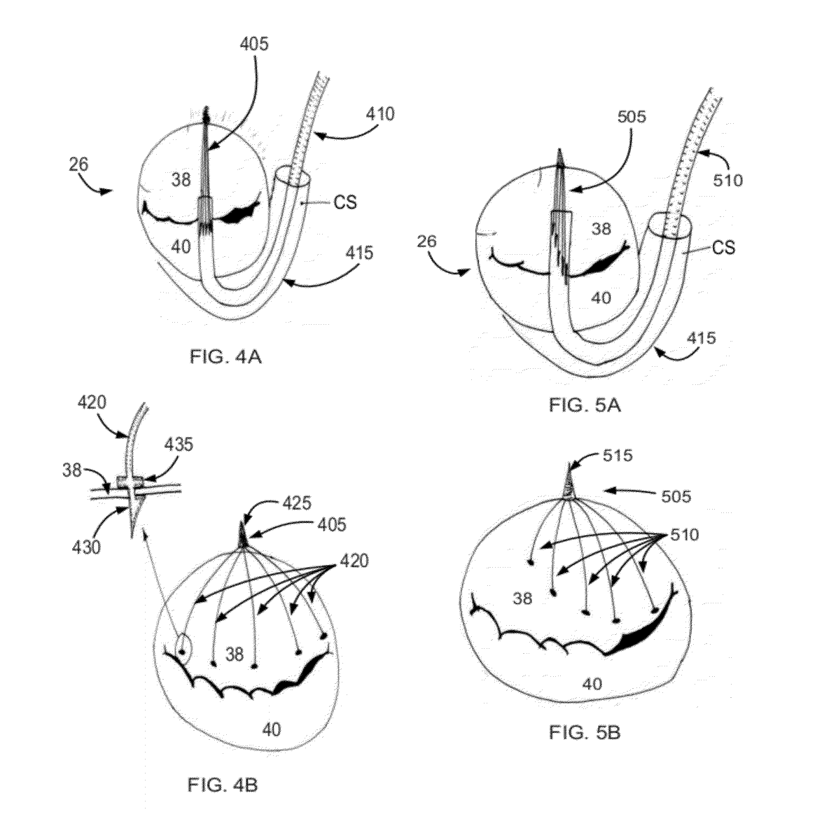

Reference is now additionally made to FIG. 4A, which is a simplified view of a first leaflet stretching device 405 constructed according to an example embodiment of the invention, just prior to engaging an anterior leaflet 38. The multi-fingered first leaflet stretching device 405 is depicted with the fingers folded against each other within a catheter 410. The catheter 410 has been inserted through the coronary sinus 415, through a wall of the left atrium 24 (FIG. 1), through the left atrium 24, and between the anterior leaflet 38 and the posterior leaflet 40 (FIG. 2A) of the mitral valve 26.

Reference is now additionally made to FIG. 4B, which is a simplified view of the leaflet stretching device 405 of FIG. 4A, after engaging the anterior leaflet 38. Equal length stretching fingers 420 are optionally each pricked into the anterior leaflet 38, and connected to a central base 425. The fingers 420 are trying to expand, distancing them from one another, thereby exerting a stretching force on the anterior leaflet 38. The leaflet 38 is now deformed, remodeled, and the lips of the anterior leaflet 38 are closer to the posterior leaflet 40, providing better coaptation.

In some embodiments of the invention, the central base 425 is embedded into the annulus of the mitral valve.

The present invention, in some embodiments thereof, includes a device which attaches to one or more locations of an atrioventricular valve leaflet, and stretches the leaflet, thereby remodeling, that is reshaping, the leaflet. Stretching members of the device are optionally positioned externally to a plane of the leaflet. The stretching fingers 420 are external to the plane of the posterior leaflet 40, with prongs attached to the stretching fingers 420 being a part of the device which does penetrate at least partially into the plane of the leaflet.

The remodeling may happen immediately as the stretching forces are applied, if the amount of stretching required is small. If the amount of stretching is larger, the stretching occurs over time.

It is noted that assessment of the result of immediate remodeling is optionally done during a transcatheter operation on a beating heart. This provides an advantage of immediate feedback to a surgeon, and possibility of correcting faults and/or improving the result during the operation.

In some embodiments of the invention one or more slits are cut into the leaflet, enabling the leaflet to stretch more immediately upon a leaflet stretching device engaging a leaflet.

In a multi-fingered stretching device, not all fingers must engage the leaflet. A surgeon may select which fingers engage the leaflet and manipulate the fingers to so engage, while optionally leaving some fingers not engaging the leaflet.

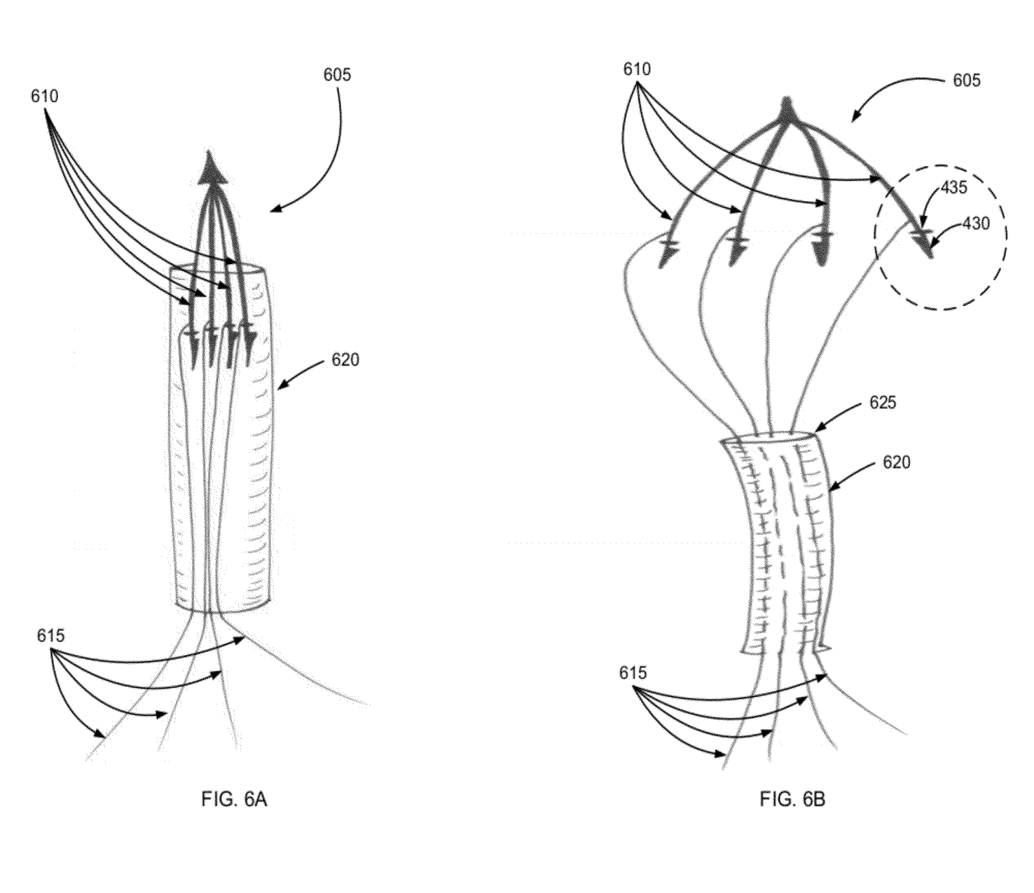

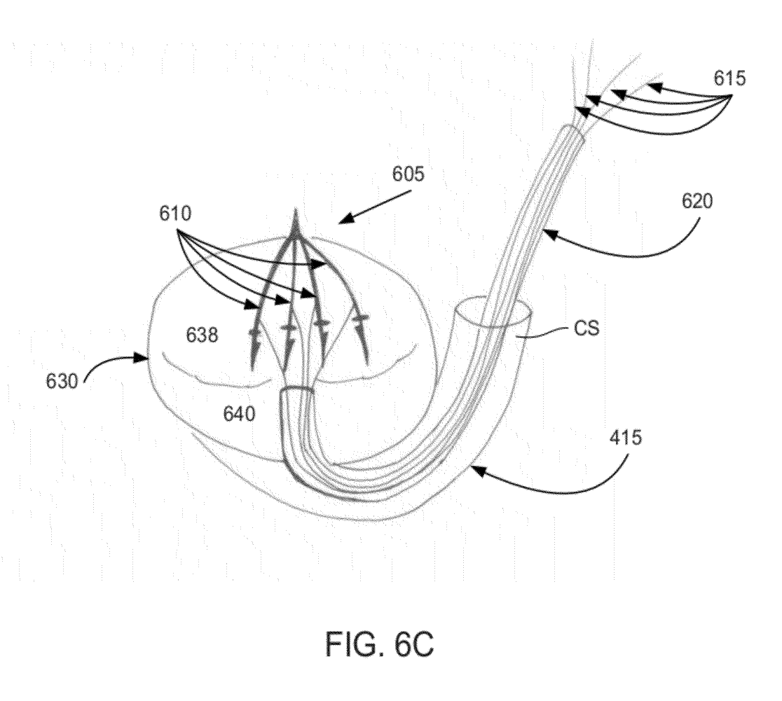

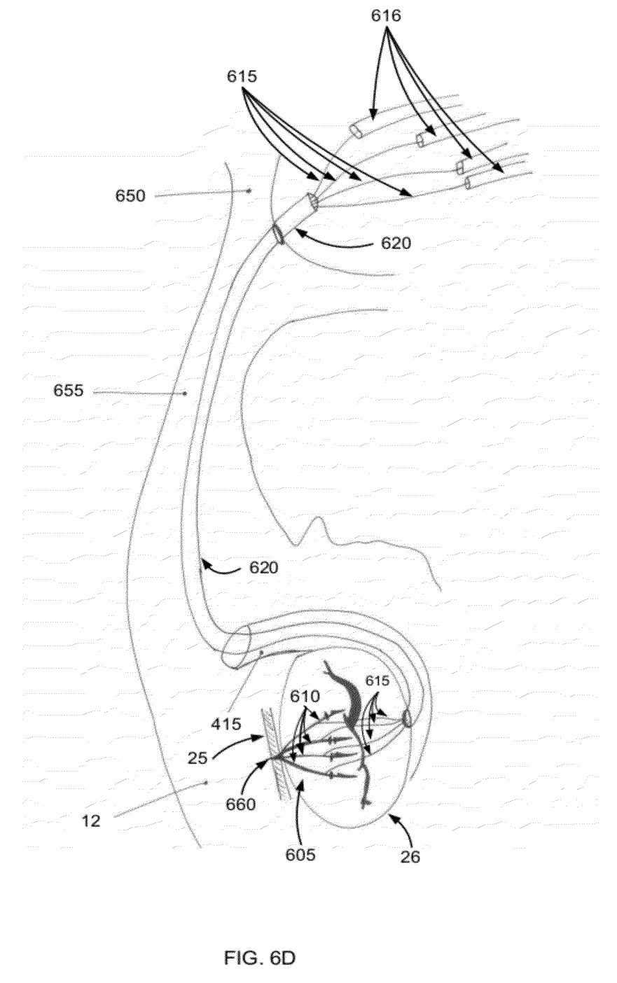

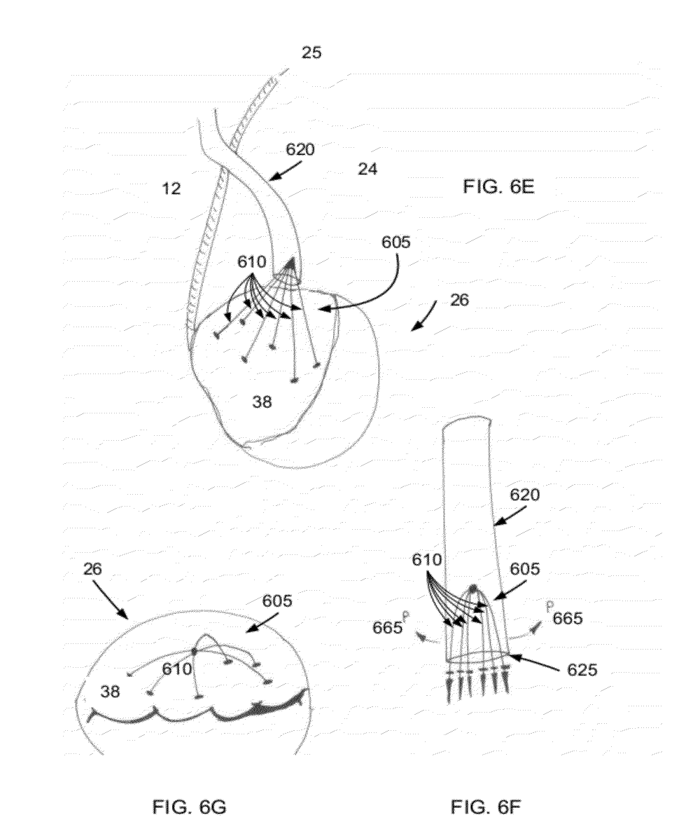

In some embodiments of the invention a catheter is inserted over guide wires attached to one or more of the stretching fingers 420, as will be further described below with reference to FIG. 6D.

In an exemplary embodiment of the invention, each finger 420 is tipped by a sharp prong 430 configured to prick through the anterior leaflet 38, and an optional collar 435 behind the prong, configured to limit an amount by which the sharp prong 430 penetrates through the anterior leaflet 38. The prong is optionally shaped as a hook in a fishing hook, also having a sharp tip pointing backward, so that the prong is hindered in pulling out of the leaflet.

Reference is now additionally made to FIG. 5A, which is a simplified view of a second leaflet stretching device 450 constructed according to another example embodiment of the invention, having different length stretching fingers, just prior to engaging an anterior leaflet 38. The multi-fingered leaflet stretching device 450 is depicted with the fingers folded against each other within a catheter 410. The catheter 410 has been inserted through the coronary sinus 415, through a wall of the left atrium 24 (FIG. 1), through the left atrium 24, and between the anterior leaflet 38 and the posterior leaflet 40 (FIG. 2A) of the mitral valve 26.

Reference is now additionally made to FIG. 5B, which is a simplified view of the leaflet stretching device 505 of FIG. 5A, after engaging the anterior leaflet 38. Different length stretching fingers 510 are each pricked into the anterior leaflet 38, and connected to a central base 515. The fingers 510 are trying to expand, distancing them from one another, thereby exerting a stretching force on the anterior leaflet 38. The anterior leaflet 38 is now deformed, remodeled, and the lips of the anterior leaflet 38 are closer to the posterior leaflet 40, providing better coaptation.

It is noted that the number of the fingers may be two and up—two fingers are needed in order to apply a stretching force. Optionally, one or more fingers may be used to engage the mitral annulus 34 (FIG. 2B), for applying a stretching force and/or for anchoring the leaflet stretching device 505. A length of the fingers is chosen to correspond to a size of the leaflet. A typical size of a finger optionally includes 0.5, 1, 2, 3, 4, and 5 cm as typical sizes, and possibly somewhat more and/or somewhat less.

It is noted that the length of each finger is optionally adapted according to size of a leaflet, and/or according to shape of a leaflet, and/or according to an amount of stretching to be applied.

It is noted that after diagnosis of a patient, optionally using medical imaging devices, a treatment planner knows where coaptation is lacking, and what portion of one or more leaflet should be stretched.

Reference is now additionally made to FIG. 6A, which is a simplified view of a third leaflet stretching device 605 constructed according to yet another example embodiment of the invention, having equal length stretching fingers 610 and having guide wires 615 attached to each finger 610.

In FIG. 6A the third leaflet stretching device 605 is folded inside a catheter. A guide wire 615 is attached to each of the stretching fingers 610, and optionally serves to control each of the stretching fingers 610, optionally control separately.

Reference is now additionally made to FIG. 6B, which is a simplified view of the third leaflet stretching device 605 of FIG. 6A.

FIG. 6B depicts the third leaflet stretching device 605, slipped slightly out of the catheter.

A tip 625 of the catheter 620 optionally constrains the guide wires 615, such that the stretching fingers 610 of the leaflet stretching device 605 optionally do not fully expand. The guide wires 615 optionally serve to separately release and allow each of the stretching fingers 610 separately to expand to a desired amount. The desired amount is optionally controlled by pulling on the guide wires 615, and optionally viewed by a practitioner using an appropriate imaging system for in-vivo imaging. Non-limiting examples of an appropriate imaging system include 3D echo and ultrasound systems.

The guide wires 615 enable pulling each of the stretching fingers 610, optionally separately, against the leaflet (not shown) so the prong 430 (also of FIG. 4B) pricks through the leaflet. The prong 430 optionally pricks into the leaflet up until the collar 435 (also of FIG. 4B).

Reference is now additionally made to FIG. 6C, which is a simplified view of the third leaflet stretching device 605 of FIG. 6A, drawn in context of an atrioventricular valve 630.

FIG. 6C depicts the third leaflet stretching device 605 partially deployed, with the stretching fingers 610 partially extended, before being slightly more extended, then pulled back toward the catheter 620 by the guide wires 615, in order to have the prongs 435 penetrate a leaflet 638 of a mitral valve 630. The catheter 620, in the example of FIG. 6C, has been inserted the coronary sinus 415.

It is noted that an additional instance or instances of a leaflet stretching device may be inserted into the heart, for example for additional stretching of the same leaflet 638.

It is noted that an additional instance or instances of a leaflet stretching device may be inserted into the heart, for example in order to remodel a different leaflet in the same valve, such as the posterior leaflet 640, and/or a leaflet in a different valve.

Reference is now additionally made to FIG. 6D, which is a simplified view of the third leaflet stretching device 605 of FIG. 6A, further having an anchor for anchoring in a heart, drawn in context of an example approach to the mitral valve 26.

An example approach for using the third leaflet stretching device 605 follows the following milestones. The catheter 620 is inserted into a right jugular vein 650, and pushed along the right jugular vein 650 to the superior vena cava (SVC) 655. The catheter 620 is further pushed along the SVC into the right atrium 12. The catheter 620 is maneuvered from the right atrium 12 into the coronary sinus 415, and pushed along the coronary sinus 415 to an appropriate point on the heart, outside the left atrium. A hole is punched through the wall of the right atrium, and the catheter is poked through the hole. At this point the tip 625 of the catheter 620 is in the right atrium, above the mitral valve 26.

The third leaflet stretching device 605 is anchored into the interatrial septum 25 by an anchor 660, and the stretching fingers 610 are each maneuvered into place by the guide wires 615.

A catheter 616 is optionally inserted along one or more of the guide wires 615.

The anchor is optionally anchored to other relatively more stable, when compared to a leaflet, parts of the heart. Such more stable parts may be, by way of some non-limiting examples, for a top approach to the mitral valve, the mitral annulus 34 and the interatrial septum 25, and for a bottom approach to the mitral valve the interventricular septum 33 and a ventricle wall. In such cases the anchor is termed an intracoronary anchor.

Reference is now additionally made to FIG. 6E, which is a simplified view of the third leaflet stretching device 605 of FIG. 6A, drawn in context of an alternative example approach to the mitral valve 26.

An alternative example approach to the mitral valve 26 follows the approach described above with reference to FIG. 6D, up until the right atrium 12. From the right atrium 12 the catheter 620 punches through the interatrial septum 25. At this point the tip 625 of the catheter 620 is in the right atrium, above the mitral valve 26.

FIG. 6E depicts the third leaflet stretching device 605 slightly extended from the catheter 620, with the stretching fingers 610 partly stretched toward the anterior leaflet 38 of the mitral valve 26.

Reference is now additionally made to FIG. 6F, which is a simplified view of the third leaflet stretching device 605 of FIG. 6A, depicting a stretching force 665 of the stretching fingers 610, controlled in part by a lip 625 of the catheter 620.

Reference is now additionally made to FIG. 6G, which is a simplified view of the third leaflet stretching device 605 of FIG. 6A, attached by the stretching fingers 610 to the anterior leaflet 38 of the mitral valve 26.

The stretching fingers 610 of the third leaflet stretching device 605 are optionally made of an elastic and/or superelastic material, so that when the stretching fingers 610 are pushed out of the catheter 620 the stretching fingers 610 assume a final shape. A surgeon optionally pushes the stretching fingers 610 slightly out of the catheter 620, engages the leaflet, then pushes the stretching fingers 610 all the way out, stretching the leaflet.

Various methods for deployment of leaflet stretching devices are contemplated. A first method includes reversed deployment of the stretching hand device, as described above with reference to FIGS. 4A-4b, 5A-5B, and 6A-6G. A second method includes using a catheter for deployment of the leaflet stretching device. A third method includes using a multi-luminal catheter for staged deployment of every stretching finger. A fourth method includes using a catheter with a moving neck, for control of expansion of the stretching fingers.

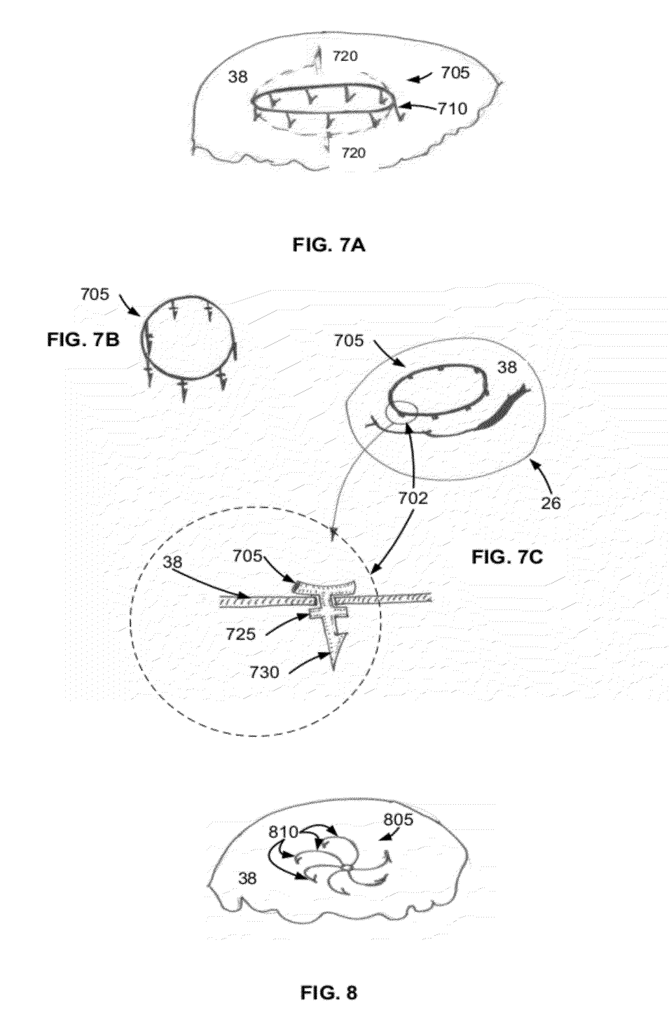

Reference is now additionally made to FIG. 7A, which is a simplified view of a fourth leaflet stretching device 705, constructed according to yet another example embodiment of the invention.

The fourth leaflet stretching device 705 has a curved shape, optionally a closed curve, with prongs along at least some of its length. The curved shape is folded when transported through a catheter (not shown), and unfolds with some force 720 when released. The fourth leaflet stretching device 705 has prongs, or hooks 710, along a circumference of the curved shape. The hooks 710 are embedded into a leaflet, by way of a non-limiting example the anterior leaflet 38 of the mitral valve. After that the curved shape of the fourth leaflet stretching device 705 is released to unfold, and exerts a stretching force 720 on the anterior leaflet 38.

Reference is now additionally made to FIG. 7B, which is a simplified view of the fourth leaflet stretching device 705, in an unfolded state.

FIG. 7B depicts the fourth leaflet stretching device 705 unfolded, as a circle. The fourth leaflet stretching device 705 reaches its unfolded shape after stretching of the anterior leaflet 38. The unfolded shape of the fourth leaflet stretching device 705 is depicted as a circle, although the unfolded shape of the fourth leaflet stretching device 705 does not necessarily have to be a circle.

Reference is now additionally made to FIG. 7C, which is a simplified view of the fourth leaflet stretching device 705, embedded in an example atrioventricular leaflet.

The fourth leaflet stretching device 705 is depicted with the hooks 710 of FIG. 7A embedded through an example atrioventricular leaflet, by way of a non-limiting example the anterior leaflet 38 of the mitral valve. The fourth leaflet stretching device 705 is depicted in a somewhat oval shape, having been embedded in a more oval, folded, state, and having somewhat stretched the anterior leaflet 38. The fourth leaflet stretching device 705 has not stretched the anterior leaflet 38 enough such that the fourth leaflet stretching device 705 reaches a circular shape such as depicted in FIG. 7B.

A magnified section 702 of FIG. 7C depicts the fourth leaflet stretching device 705, and one prong embedded in the anterior leaflet 38. The ring of the fourth leaflet stretching device 705 is optionally embedded flush with the anterior leaflet 38, with a sharp tip 730 penetrating through the anterior leaflet 38, and a collar 725 penetrating through the anterior leaflet 38 and preventing the sharp tip 730 from pulling back through the anterior leaflet 38.

Reference is now additionally made to FIG. 8, which is a simplified view of a fifth leaflet stretching device 805, constructed according to yet another example embodiment of the invention.

The fifth leaflet stretching device 805 includes spiral stretching fingers 810 extending outward from a central ring. When in a catheter, the fifth leaflet stretching device 805 is folded, and when embedded in an atrioventricular leaflet, such as, by way of a non-limiting example, the anterior leaflet 38 of the mitral valve 26, the spiral stretching fingers 810 expand, stretching the anterior leaflet 38.

The spiral stretching fingers 810 provide a non-limiting example of an arrangement of fingers which are elastic and exert a stretching force on the leaflet in which the fingers are engaged. Other non-limiting examples include zigzag shaped fingers, fingers which include prongs attached to springs, and so on.

It is noted that the fingers do not have to be attached to a central ring, and may extend from a common center without a central ring.

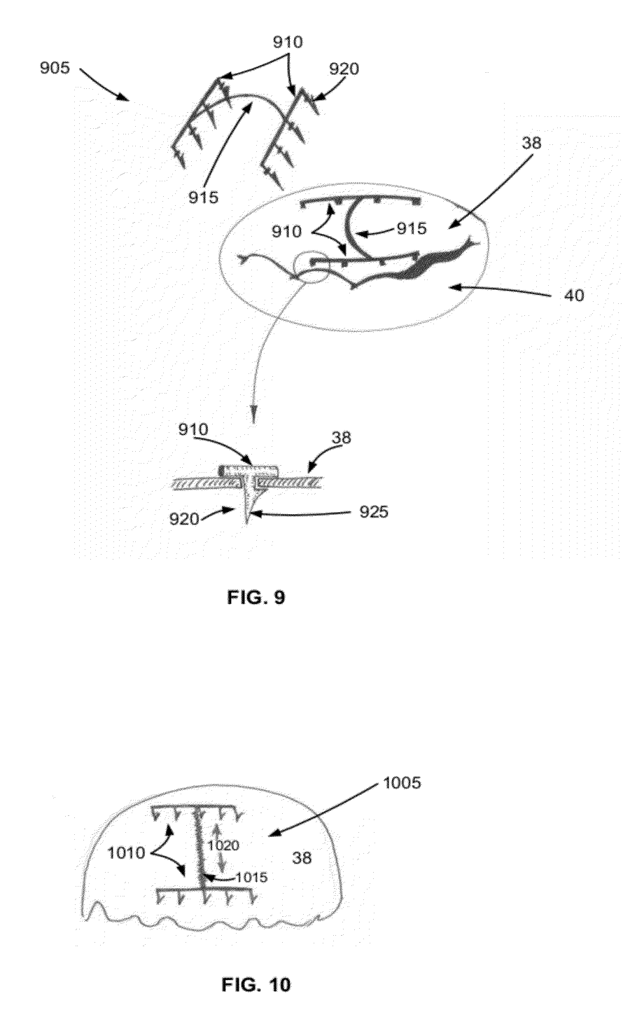

Reference is now additionally made to FIG. 9, which is a simplified view of a sixth leaflet stretching device 905, constructed according to yet another example embodiment of the invention.

The sixth leaflet stretching device 905 includes two rows of prongs 920, each row of prongs connected to a connecting member 910, and a bow-shaped spring 915 between the connecting members 910. The sixth leaflet stretching device 905 is optionally transferred via a catheter (not shown), with the bow-shaped spring 915 folded. The sixth leaflet stretching device 905 is optionally extended from the catheter, with the bow-shaped spring 915 still folded and under tension, at which time prongs 920 are embedded into an atrioventricular valve leaflet, by way of a non-limiting example the mitral valve anterior leaflet 38.

It is noted that the prongs 920 are optionally folded while inside a catheter (not shown)

It is noted that the bow shaped spring 915 is an example of a device for pushing the connecting members 910 apart, and that other shapes may serve the same purpose.

A magnified section of FIG. 9 depicts a prong 920 embedded in the anterior leaflet 38, with a tip of the prong constructed so as to prevent the tip 925 of the prong 920 from backing back up through the anterior leaflet 38, and the body of the sixth leaflet stretching device 905 optionally abutting against the anterior leaflet 38, performing a function of the collar 435 of FIG. 4B.

It is noted that while FIG. 9 depicts two rows of prongs 920, and two connecting members 910, three or more rows of prongs 920 and connecting members 910 can optionally be used. The number of rows of prongs 920 is optionally selected based upon a desired stretching effect upon a leaflet.

It is noted that the rows of prongs 920 are not necessarily straight. In some embodiment the rows of prongs 920 are each a segment of an arc. Other shapes are also contemplated, to suit grasping a desired portion of a valve leaf, and/or to suit embedding as an anchor in the valve annulus and/or heart wall.

Reference is now additionally made to FIG. 10, which is a simplified view of a seventh leaflet stretching device 1005, constructed according to yet another example embodiment of the invention.

The seventh leaflet stretching device 1005 includes two rows of prongs, each connected to a connecting member 1010, and a coil spring 1015 between the two connecting members 1010. The seventh leaflet stretching device 1005 is optionally transferred via a catheter (not shown), with the coil spring 1015 compressed. The seventh leaflet stretching device 1005 is optionally extended from the catheter, with the coil spring 1015 still compressed and under tension, at which time prongs are embedded into an atrioventricular valve leaflet, by way of a non-limiting example the mitral valve anterior leaflet 38.

In some embodiments of the invention the spring 1015 is held compressed and under tension by a dissolvable sugar or a dissolvable plastic, which slowly dissolve in the body. After dissolving, the spring 1015 exerts more force on the leaflet.

When the seventh leaflet stretching device 1005 is optionally embedded into the anterior leaflet 38, the coil spring 1015 exerts a force 1020 which stretches the anterior leaflet 38.

In some embodiments, one of the connecting members 1010 is embedded in the mitral valve annulus.

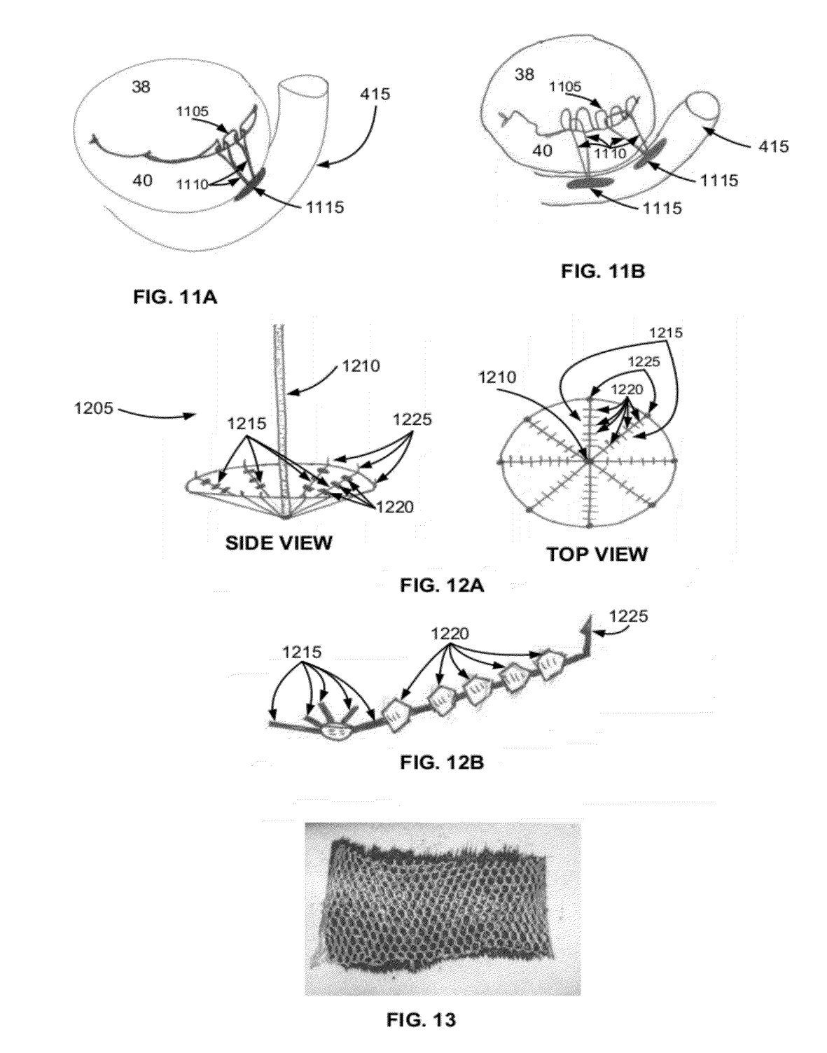

Reference is now additionally made to FIGS. 11A and 11B, which are simplified views of a leaflet stretching device 1105 anchored through a left atrium wall to the coronary sinus 415.

In some cases it is desired that the leaflet stretching device 1105 be anchored to an additional location. Reasons for anchoring include an additional pulling on the leaflet, in order to additionally remodel the leaflet, and attaching the leaflet stretching device 1105 to an additional location so that it may not sweep away, even if detached from the leaflet.

FIG. 11A depicts an embodiment of a leaflet stretching device 1105, attached to a mitral valve anterior leaflet 38, and anchored by one or more tethers 1110, to a location in the left atrium, which penetrate into the left atrium wall (not shown) and optionally even through the left atrium wall into the coronary sinus 415 (as shown). The tethers 1110 are optionally attached to an anchor 1115 in the coronary sinus 415. The anchoring is optionally performed as part of placing the leaflet stretching device 1105, when the approach to the mitral valve was an approach through the coronary sinus 415.

FIG. 11B depicts an embodiment of a leaflet stretching device 1105, attached to a mitral valve anterior leaflet 38, and anchored by one or more tethers 1110, to two locations in the left atrium, which penetrate into the left atrium wall (not shown) and optionally even through the left atrium wall and into the coronary sinus 415 (as shown). The tethers 1110 are optionally attached to an anchor 1115 in the coronary sinus 415. The location of at least one of the anchors is optionally the hole formed by the approach to the mitral valve through the coronary sinus 415.

It is noted that the position of the anchors of FIG. 11B may be spaced further apart than depicted in FIG. 11B, by way of a non-limiting example one on each side of the left atrium.

It is noted that the anchor or anchors may be in the ventricle, if the leaflet stretching device 1105 is engaged with the lower, ventricle, side of the leaflet.

Reference is now additionally made to FIGS. 12A and 12B, which are simplified views of an eighth leaflet stretching device 1205, constructed according to yet another example embodiment of the invention.

The eighth leaflet stretching device 1205 includes an umbrella-shaped part, including a stem 1210 and ribs 1215. Along the ribs 1215 are attached blades 1220 to slit partway through, and/or all the way through, a leaflet.

A slit which penetrates partway through, and cuts through lamina and connective tissue of the leaflet without cutting all the way through, is considered to be good for stretching the leaflet.

Making the slits in the leaflet enables the leaflet to expand and remodel more than a leaflet without slits. Making the slits in the leaflet also provides control for into what shape the leaflet will remodel, more than a leaflet without slits.

Optionally, at ends of the ribs 1215 are placed prongs 1225.

The eighth leaflet stretching device 1205 optionally has the blades 1220 and the prongs 1225 pointed back along the stem 1210, making the eighth leaflet stretching device 1205 suitable for a reverse embedding, such as penetrating through a atrioventricular valve, opening the umbrella structure, and reversing the eighth leaflet stretching device 1205 to have the blades slit the leaflet and the prongs 1225 penetrate the leaflet.

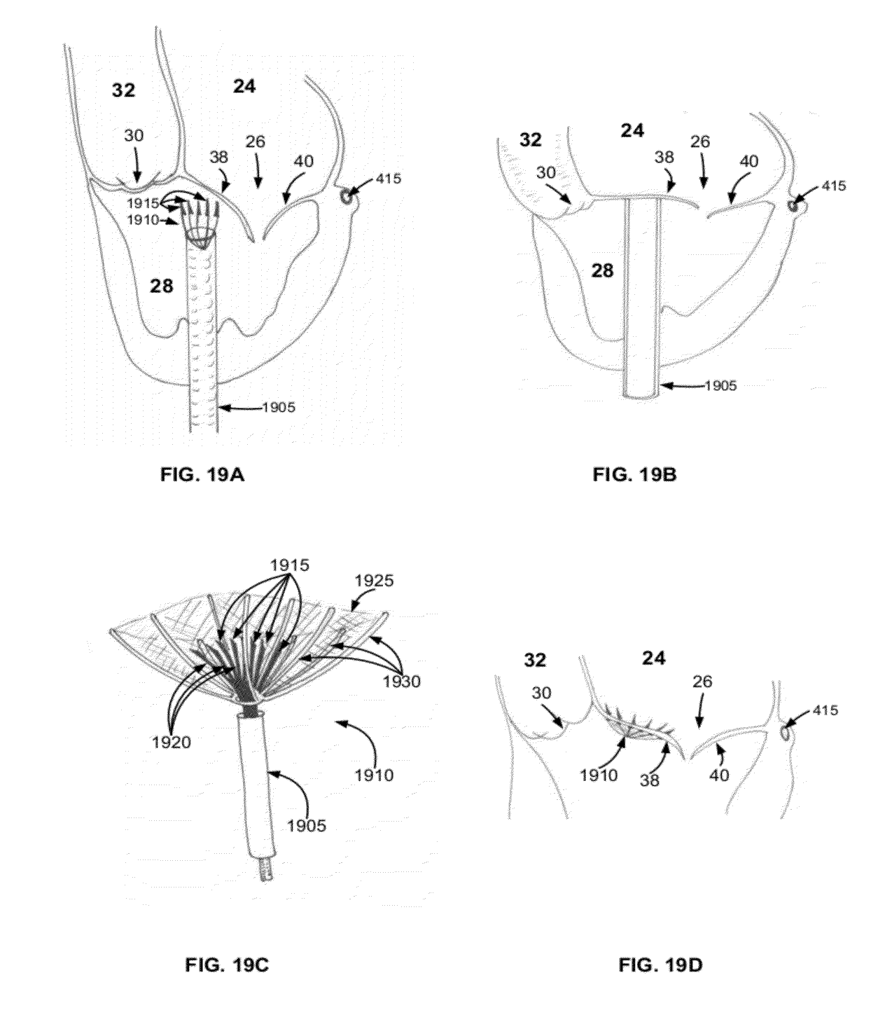

An additional method for having the stem 1210 poke through the leaflet is by using a transapical approach (see description for FIGS. 19A-19D below), and pushing the stem 1210 through the leaflet. The stem 1210 may optionally be pushed against the leaflet while the leaflet is optionally braced against an inflatable balloon supporting the leaflet from behind.

The umbrella structure of the eighth leaflet stretching device 1205 is optionally transported folded through a catheter (not shown). The eighth leaflet stretching device 1205 is pushed out of the catheter, and optionally opened somewhat. The eighth leaflet stretching device 1205 is then pulled back onto the leaflet, the prongs 1225 embed in the leaflet, and the blades 1220 slit the leaflet. The prongs 1225 may be maneuvered to embed first and the blades 1220 maneuvered to slit after, or the opposite may be maneuvered, such that the blades 1220 are maneuvered to slit first and the prongs 1225 are maneuvered to embed after, or the maneuver may optionally be performed substantially simultaneously. After slitting and prong penetration, the umbrella structure is optionally further expanded, stretching the leaflet and remodeling the leaflet. It is noted that the remodeling may happen immediately with the stretching, and/or over a period of time.

In some embodiments of the invention, blade depth is limited, and cutting slits in the leaflet is performed such that the slits do not cut all through the leaflet. In some embodiments of the invention, a stop (not shown) is built into each blade, such that cutting slits in the leaflet is limited, and the slits do not cut all through the leaflet.

In some embodiments of the invention, cutting slits in the leaflet is optionally performed such that the slits do cut all through the leaflet, making holes in the leaflet. In such embodiments, a mesh is optionally placed over the open slits, so that the mesh reduces and/or blocks flow of blood through the slits. The mesh also optionally, when constructed of suitable material, such as low porosity or optionally zero porosity material, helps the slits mend faster.

Reference is now additionally made to FIG. 13, which is a simplified image of a mesh 1305 for optional use with a leaflet stretching device which cuts holes through a leaflet.

It is noted that the mesh 1305 is optionally flexible in a bending direction, that is, perpendicular to a surface of the mesh, and/or optionally flexible for stretching, that is along the surface of the mesh.

In some embodiments of the invention the mesh 1305 is chosen so as to catch on the prongs.

It is noted that the mesh described with reference to FIGS. 13, 16, 17G, 17H, 18, 19C, 21, and 23C is expected to optionally adhere to a leaflet by a coagulating of punctures in the leaflet. The adherence of the mesh to the leaflet assists in preventing possible tearing of the leaflet.

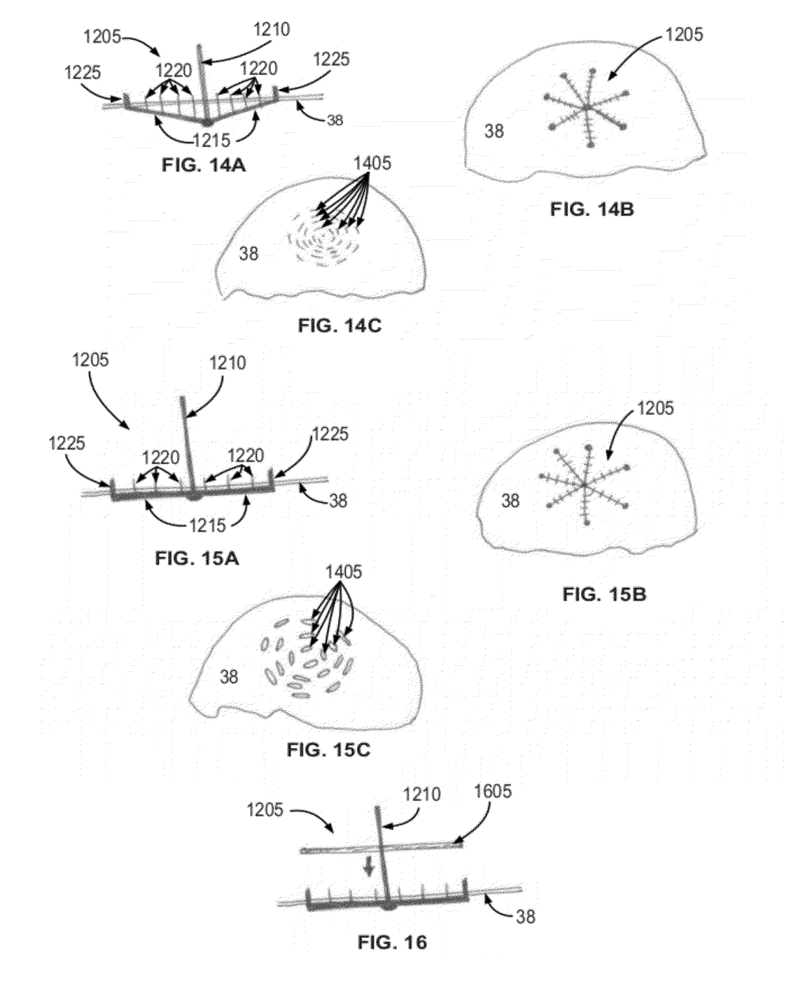

Reference is now additionally made to FIGS. 14A, 14B, and 14C, which are simplified views of the eighth leaflet stretching device 1205 of FIG. 12A partially open and embedded into an atrioventricular valve leaflet.

FIG. 14A depicts a side view of the eighth leaflet stretching device 1205 with the stem 1205 pushed through a leaflet, by way of a non-limiting example the mitral valve anterior leaflet 38. The eighth leaflet stretching device 1205 is in a partially open state, with the prongs 1225 embedded in the anterior leaflet 38, and the blades 1220 cutting into the anterior leaflet 38.

FIG. 14B depicts a top view of the eighth leaflet stretching device 1205 in the same state as FIG. 14A.

FIG. 14C depicts a top view of the anterior leaflet 38, without depicting the eighth leaflet stretching device 1205, showing slits 1405 cut into the anterior leaflet 38.

Reference is now additionally made to FIGS. 15A, 15B, and 15C, which are simplified views of the eighth leaflet stretching device 1205 of FIG. 12A fully open and embedded into an atrioventricular valve leaflet.

FIG. 15A depicts a side view of the eighth leaflet stretching device 1205 with the stem 1205 pushed through and stretching the anterior leaflet 38. The eighth leaflet stretching device 1205 is in a fully open state, with the prongs 1225 embedded in and stretching the anterior leaflet 38.

FIG. 15B depicts a top view of the eighth leaflet stretching device 1205 in the same state as FIG. 15A.

FIG. 15C depicts a top view of the anterior leaflet 38, without depicting the eighth leaflet stretching device 1205, showing the slits 1405 cut into the anterior leaflet 38, now stretched to have a substantially oval shape.

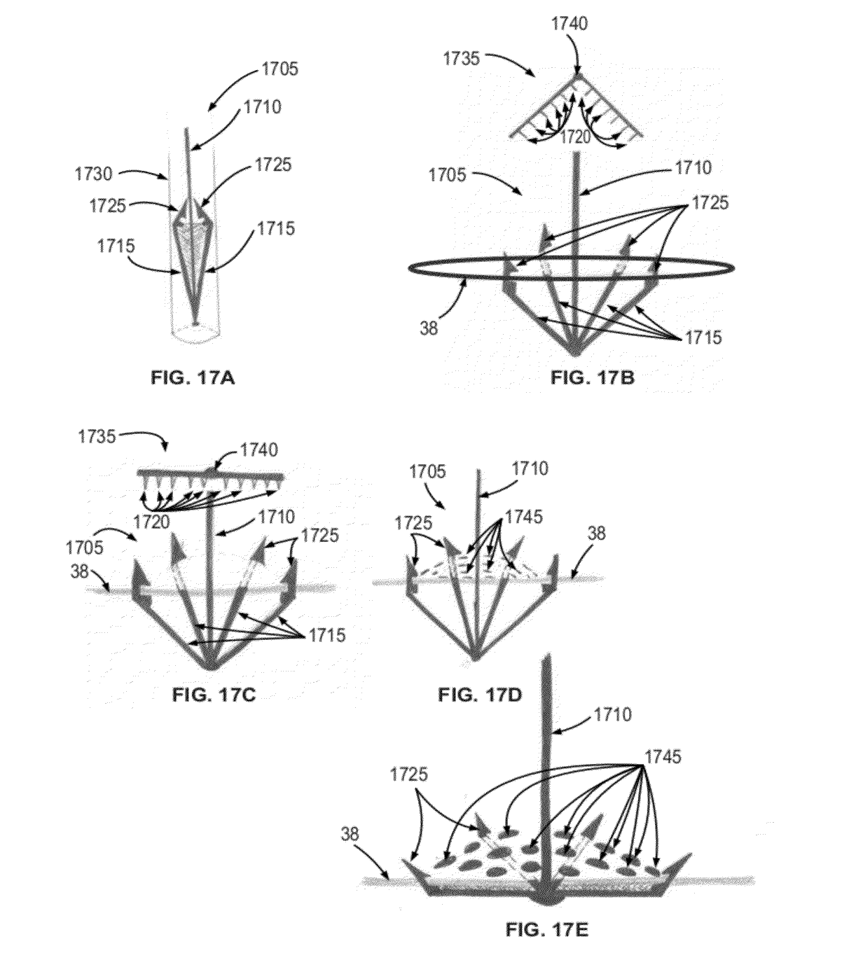

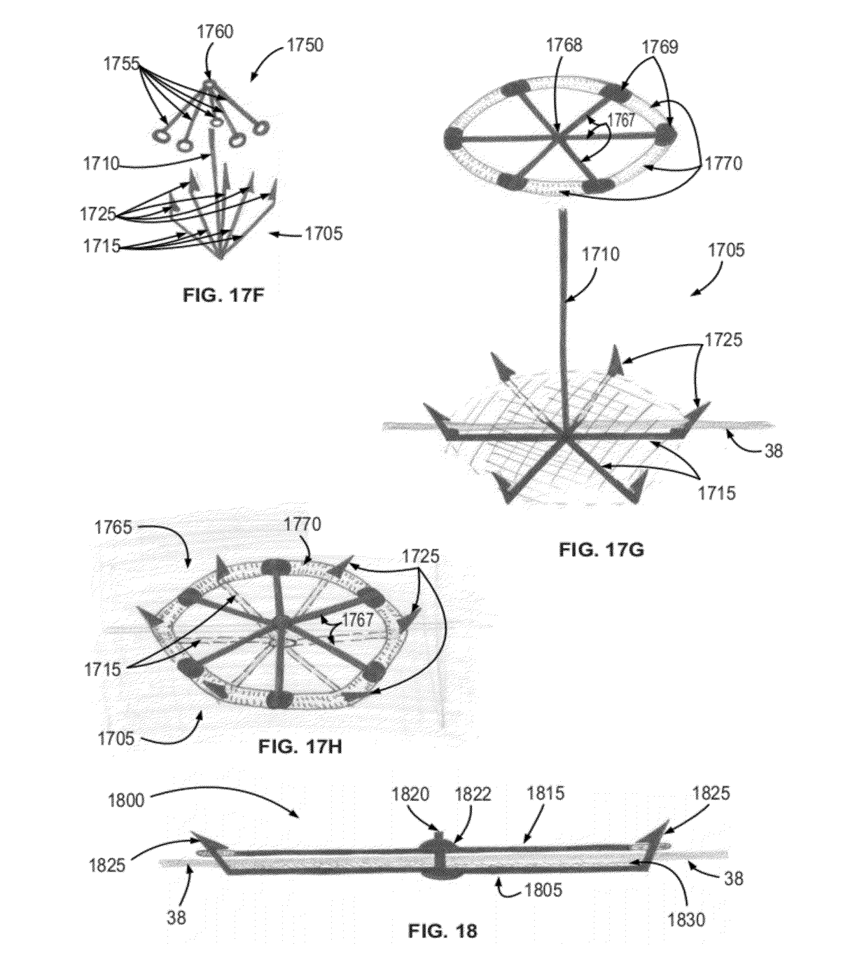

In some embodiments of the invention the cutting of the slits is optionally performed by a separate tool, as described below with reference to FIGS. 17A-17E.

Reference is now additionally made to FIG. 16, which is a simplified view of the eighth leaflet stretching device 1205 of FIG. 12A fully open and embedded into an atrioventricular valve leaflet, and an optional mesh 1605 closing upon an opposite side of the atrioventricular valve leaflet.

FIG. 16 depicts the eighth leaflet stretching device 1205 in a state, relative to the anterior leaflet 38, similar to that depicted in FIG. 15A. In addition, FIG. 16 depicts an optional mesh 1605 being slid along the stem 1210, to abut against the anterior leaflet 38. The optional abutting mesh 1605 optionally serves for reducing blood leakage through the slits 1405 of FIGS. 14C and 15C, and/or for additionally adjusting the shape of the anterior leaflet 38.

In some embodiments of the invention the mesh limits and/or controls reshaping. In order to control reshaping the mesh is optionally made of memory material, which is optionally inserted into place having a first shape, and takes on a second shape after being inserted in place. Non-limiting examples of memory materials include:

1. Shape memory alloys and/or shape memory polymers, which include thermo-responsive materials where deformation can be induced and recovered through temperature changes.

2. Magnetic shape memory alloys, which include materials which change their shape in response to a significant change in the magnetic field.

3. pH-sensitive polymers, which include materials which swell/collapse when a pH of the surrounding media changes.

In some embodiments of the invention the mesh prevents overstretching. In some embodiments, in order to prevent overstretching the mesh is optionally woven of non-stretchable material, in which the pattern in which the non-stretchable material is woven allows stretching up to a limit, beyond which the non-stretchable woven material does not allow stretching. In some embodiments, in order to prevent overstretching the mesh is optionally woven of a material which resists stretching with a force which increases the more the material stretches. A sum of a resistance of the leaflet to stretching plus the resistance of the material of the mesh to stretching overcomes the stretching of the stretching device, once the material of the mesh has been stretched by a specific amount. The specific amount of stretching is optionally measured on a sample mesh, learning the force by which a mesh resists stretching by measuring the force on a sample mesh substantially identical to a mesh intended for use in a leaflet.

In some embodiments of the invention the mesh affects stretching in one direction differently than on another direction, thereby affecting the reshaping.

Reference is now additionally made to FIGS. 17A to 17H, which are simplified views a ninth leaflet stretching device 1700, constructed according to yet another example embodiment of the invention, and operation of the ninth leaflet stretching device 1700 in context of an atrioventricular valve leaflet.

The several embodiments of the ninth leaflet stretching device 1700 include an umbrella shaped portion 1705, depicted in FIGS. 17A-17H, an additional foldable umbrella of blades 1735, depicted in FIGS. 17B and 17C, and optionally an additional counter umbrella 1750, several embodiments of which are depicted in FIGS. 17F-17H.

Operation of the ninth leaflet stretching device 1700 in context of an atrioventricular valve leaflet is depicted in order, from FIG. 17A to FIG. 17H.

FIG. 17A depicts the umbrella shaped portion 1705 inserted in a catheter 1730, for transport in vivo. The umbrella shaped portion 1705 includes stretching fingers 1715 with prongs 1725 at ends of the stretching fingers 1715, folded against a stem 1710. The umbrella shaped portion 1705 is constructed so the stretching fingers 1715 may unfold and open into a shape of an open umbrella.

FIG. 17B depicts the umbrella shaped portion 1705 outside of the catheter (not shown), and partially open. The umbrella shaped portion 1705 is in a partially open state, with the stretching fingers 1715 partially spread out, and with the prongs 1725 embedded through an atrioventricular valve leaflet, such as the mitral valve anterior leaflet 38.

The foldable umbrella of blades 1735 is optionally transferred to the location of the umbrella shaped portion 1705, either through the same catheter or through an additional catheter. The foldable umbrella of blades 1735 is depicted partially unfolded, showing a plurality of blades 1720, similar to the blades 1220 of FIG. 12A. The foldable umbrella of blades 1735 includes a center hole 1740 configured to optionally fit onto and slide along the stem 1710 of the umbrella shaped portion 1705.

A non-limiting example of a diameter of the partially open state of the umbrella shaped portion 1705 is about 12 mm. The foldable umbrella of blades 1735, when unfolded, is correspondingly typically of a diameter somewhat less than the 12 mm. The umbrella of blades 1735 cuts slits into the leaflet, after which the umbrella shaped portion 1705 expands to a larger diameter, such as, by way of a corresponding and non-limiting example, 20 mm, exerting a stretching force on the leaflet.

In some embodiments of the invention, the umbrella of blades 1735 cuts slits into the leaflet over an area comprising about 20% to 80% of the area of the leaflet.

In experiments, an enlargement of the leaflet's area of greater than 150 mm² has been achieved.

In some embodiments of the invention an optional locking mechanism is also included in the foldable umbrella of blades 1735, optionally close to and/or included in the center hole 1740.

FIG. 17C depicts the umbrella shaped portion 1705 embedded in the anterior leaflet 38, and the foldable umbrella of blades 1735 unfolded and located so that the center hole 1740 of the umbrella of blades 1735 is ready to be slide along the stem 1710 of the umbrella shaped portion 1705.

The umbrella of blades 1735 is slid along the stem 1710 of the umbrella shaped portion 1705, cutting slits into the anterior leaflet 38 with the blades 1720.

The slits may optionally be cut to be longer than the blades 1720, optionally by maneuvering the blades 1720 in cutting motions.

After cutting the slits, the umbrella of blades 1735 is optionally withdrawn from the anterior leaflet 38, from the stem 1710 of the umbrella shaped portion 1705, optionally folded, and withdrawn from the body.

FIG. 17D depicts the umbrella shaped portion 1705 with the prongs 1725 embedded into the anterior leaflet 38, and slits 1745 cut into the anterior leaflet 38.

The umbrella shaped portion 1705 is then opened further. The prongs 1725 pull on the anterior leaflet 38, stretching the anterior leaflet 38, and widening the slits 1745.

FIG. 17E depicts the umbrella shaped portion 1705 opened further, with the prongs 1725 embedded into the anterior leaflet 38 and stretching the anterior leaflet 38, and the slits 1745 wider than depicted in FIG. 17D.

In cases where the slits do not cut through the anterior leaflet 38, the above may be an end to the operation of the ninth leaflet stretching device 1700.

An optional continuation of the operation is performed by inserting an additional counter umbrella 1750. The counter umbrella 1750 has been optionally transferred to the location of the umbrella shaped portion 1705, either through the same lumen in a catheter as the umbrella shaped portion 1705, or through the same lumen as the umbrella of blades 1735, or through an additional lumen.

The counter umbrella 1750 is optionally folded while in transport through a catheter, and may be opened upon arriving on location and exiting the catheter.

FIG. 17F depicts the counter umbrella 1750 having a plurality of spokes 1755, partially open, placed near the umbrella shaped portion 1705, located so as to be able to slide along the stem 1710. A center 1760 of the counter umbrella 1750 includes a hole shaped to slide along the stem 1710 of the umbrella shaped portion 1705. The spokes 1755 optionally serve for keeping a leaflet, such as the mitral valve anterior leaflet 38 (not shown in FIG. 17F) against the umbrella shaped portion 1705, assisting the reshaping of the leaflet.

In some embodiments of the invention an optional locking mechanism is also included in the counter umbrella 1750, optionally close to and/or included in the center 1760.

FIG. 17G depicts a second embodiment of a counter plate 1765, having a plurality of tines 1767, a center 1768 having a hole for sliding along the stem 1710 of the umbrella shaped portion 1705, and a mesh 1770 connecting at least between tips 1769 of the tines 1767.

In operation, the center 1760 of the counter umbrella 1750 or the counter plate 1765 is slid along the stem 1710 of the umbrella shaped portion 1705, until the counter umbrella 1750 abuts the anterior leaflet 38.

It is noted that the number of stretching fingers 1715 may vary. Six stretching fingers 1715 is a number which has been found useful, while the range of 5-8 is considered to be substantially similarly useful.

FIG. 17H depicts the counter plate 1765 abutting the anterior leaflet 38 against the umbrella shaped portion 1705, with the mesh 1770 covering those locations where the prongs 1725 of the umbrella shaped portion 1705 have pierced through the anterior leaflet 38. The mesh 1770 optionally helps prevent leakage through holes which the prongs 1725 pierced in the anterior leaflet. The tines 1767 are optionally rotated about the stem 1710 so as to be between the stretching fingers 1715, optionally helping to remodel the anterior leaflet 38.

Reference is now additionally made to FIG. 18, which is a simplified view of a tenth leaflet stretching device 1800, constructed according to yet another example embodiment of the invention, in context of an atrioventricular valve leaflet.

In the tenth leaflet stretching device 1800, a mesh 1830 is included in an umbrella shaped portion 1805. The umbrella shaped portion 1805 is depicted already open, similarly to FIGS. 17E, 17G, and 17H, with prongs 1825 already embedded in the atrioventricular valve leaflet, again depicted by way of a non-limiting example as mitral valve anterior leaflet 38. A counter plate 1815 is depicted abutting the anterior leaflet 38, and a fastener 1822 is fastened to a stem 1820 of the umbrella shaped portion 1805 locking the counter plate 1815 into place against the umbrella shaped portion 1805.

A description is now made, with reference to FIGS. 19A-19C, 20, and 21, of several example approaches by which a leaflet stretching device may be brought through a body to an atrioventricular valve.

It is noted that FIGS. 6D and 11 above have already described an approach through the coronary sinus, and FIG. 6E above has described a trans-septum approach through the interatrial septum.

Reference is now additionally made to FIGS. 19A to 19D, which are simplified views of a bottom, transapical, approach to a mitral valve 26 and an example embodiment of a leaflet stretching device 1910.

It is noted that the above mentioned approach can be used with any embodiment of the leaflet stretching devices described herein.

FIG. 19A depicts a catheter 1905 inserted through a bottom of a left ventricle 28, and deploying an embodiment of a leaflet stretching device 1910. Having inserted the catheter 1905 through the bottom of the left ventricle 28, the leaflet stretching device 1910 is positioned right below the mitral valve 26, and prongs 1915 are positioned to penetrate either an anterior leaflet 38 of the mitral valve 26, or a posterior leaflet 40 of the mitral valve 26.

FIG. 19B depicts an optional use for a catheter 1905. The catheter 1905 may optionally be used to control, that is, for example, stabilize and/or immobilize, a leaflet, by way of a non-limiting example an anterior leaflet 38 of a mitral valve 26, by using suction to pull the anterior leaflet 38 against the catheter 1905, before deploying the leaflet stretching device 1910. Controlling a leaflet enables a surgeon to know where the leaflet is, most especially when working in a beating heart.

FIG. 19C depicts an example embodiment of a leaflet stretching device 1910 in more detail than FIG. 19A. The leaflet stretching device 1910 may be deployed, folded, through a catheter 1905. The embodiment of FIG. 19C depicts stretching fingers 1920 with prongs 1915 for embedding into a leaflet, and a mesh 1925 with tines 1930 folded around the stretching fingers 1920 and enveloping the stretching fingers 1920.

FIG. 19D depicts a result of embedding a leaflet stretching device 1910 in an anterior leaflet 38 of a mitral valve 26.

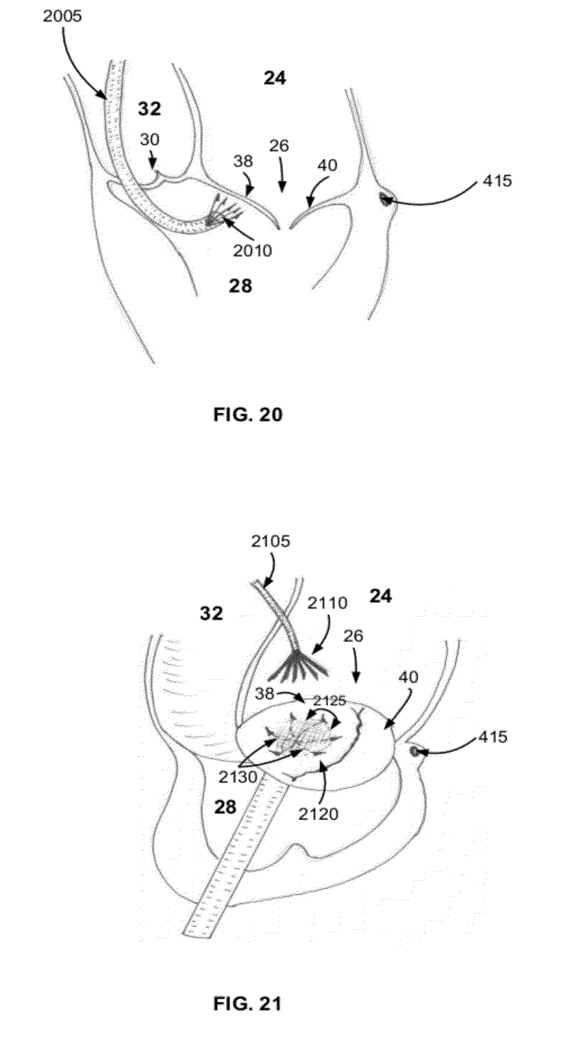

Reference is now additionally made to FIG. 20, which is a simplified view of another bottom approach to a mitral valve 26.

A catheter 2005 is inserted into the aorta 32, by any method presently known in the art. The catheter 2005 is pushed up the aorta 32, past a semi-lunar valve 30, into a left ventricle 28. When in the left ventricle 28, the catheter 2005 is positioned below a mitral valve 26, in position for embedding a leaflet stretching device 1910 in any one of an anterior leaflet 38 of the mitral valve 26 or a posterior leaflet 40 of the mitral valve 26.

Reference is now additionally made to FIG. 21, which is a simplified view of combined top and bottom approaches to a mitral valve 26.

A first catheter 2105 is inserted into the aorta 32, by any method presently known in the art. The first catheter 2105 penetrates a wall between the aorta 32 and a left atrium 24, in position above a mitral valve 26, for embedding a leaflet stretching device 2110 in any one of an anterior leaflet 38 of the mitral valve 26 or a posterior leaflet 40 of the mitral valve 26. FIG. 21 depicts the leaflet stretching device 2110 embedded, by way of a non-limiting example, in the anterior leaflet 38 of the mitral valve 26.

A second catheter 2115 is inserted through a bottom of a left ventricle 28, and optionally deploys an embodiment of a counter plate 2120, optionally including a mesh 2125 and optionally tines 2130 for optionally shaping the mesh 2125 and optionally remodeling the anterior leaflet 38.

It is noted that both presently conventional and presently non-conventional approaches are optionally used for leaflet stretching device application. Non-limiting example approaches include: a transfemoral and a transjugular vein approach with transseptal punction; a transaortic retrograde approach; a transcoronary sinus approach; and a transapical approach by back punction.

It is noted that two or more of the approaches may be used in the same procedure.

A description is now made of several non-limiting example embodiments of prongs, constructed according to the present invention, with which a leaflet stretching device may engage a leaflet.

Reference is now additionally made to FIGS. 22A-22E, which are simplified drawings of example prongs constructed according to the present invention.

The drawings include sections from previous drawings depicting embodiments of the invention.

The term “prong” in all its grammatical forms is used throughout the present specification and claims to mean a “leaflet engaging element”. Additionally, below, with reference to FIG. 22E, an additional form of a leaflet engaging element will be described.

FIG. 22A corresponds to a lower half of FIG. 17F, depicting stretching fingers 1715 having prongs 1725 with sharp tips for inserting into a leaflet. When the prongs 1725 are inserted they may be at a close-to-perpendicular angle to a surface of the leaflet (not shown), and the stretching force may be at a close-to-parallel angle to the surface of the leaflet. The stretching force therefore does not substantially cause the prongs 1725 to further penetrate the surface of the leaflet.

In some embodiments the tips of the prongs may reach an angle which is close-to-parallel to the surface of the leaflet, in which case the stretching force may cause the prongs to be further inserted into the leaflet and slide through the leaflet instead of stretching the leaflet.

It is noted that a tip of the prong is optionally shaped like a fish-hook, to optionally hinder a prong which has penetrated a leaflet from sliding back out of the leaflet. The prong tip may be simply sharpened, without the extra fish-hook-like feature, but such a prong is not depicted in FIGS. 22A-22E.

FIG. 22B corresponds to an enlarged section of FIG. 4B, depicting a prong 430 penetrating through an anterior leaflet 38, and having a collar 435 behind the prong 430, to hinder a stretching fingers 420 from sliding through the anterior leaflet 38 instead of engaging the anterior leaflet.

FIG. 22C corresponds to an enlarged section of FIG. 9, depicting a prong 920 with a tip 925 penetrating through an anterior leaflet 38. The leaflet stretching device of FIG. 9 has a connecting member 910 which prevents the prong 925 from further penetrating through the anterior leaflet 38.

FIG. 22D corresponds to an enlarged section of FIG. 7C, depicting a sharp tip 730 of a penetrating prong, having a small collar 725 which also penetrates through the anterior leaflet 38. The fourth leaflet stretching device 705, which is depicted in FIG. 7C, lies along the anterior leaflet 38 and prevents additional penetration or sliding through the leaflet. The small collar 725 penetrates through the anterior leaflet 38, and hinders the sharp tip 730 of the prong from sliding back out of the anterior leaflet 38.

FIG. 22E corresponds to FIG. 12B, having blades 1220 which penetrates and cut into a leaflet (not shown). The blades 1220 are attached to ribs 1215 of an umbrella-like structure, which expands. The blades 1220 cut along their edges, and then exert a stretching force when the umbrella is extended. The blades 1220 act both for cutting, and, in their function of exerting a stretching force, as leaflet engaging elements. The stretching force is substantially perpendicular to the blade edge, therefore does not substantially cut the leaflet.

It is noted that a leaflet stretching device may include a combination of prongs and blades. Indeed, an optional prong 1225 is depicted in FIG. 22E, at a tip of a rib 1215.

It is noted that a plurality of leaflet engaging elements are optionally used in each leaflet stretching device. It is possible that one or more leaflet engaging elements might not engage a leaflet, and/or that one or more leaflet engaging elements work loose of the leaflet. Nevertheless, other one or more leaflet engaging elements will continue to engage the leaflet, thereby preventing the leaflet stretching device from working loose.

Some embodiments of the invention have 8 or more leaflet engaging elements, making a scenario of the leaflet stretching device working loose vey unlikely.

It is noted that having a plurality of leaflet engaging elements spreads stretching force over a plurality of engaging locations, exerting a limited stretching force in each of the engaging locations. Limiting the stretching force is intended to prevent leaflet or chordal rupture. Additional methods for preventing leaflet or chordal rupture include not operating on patients with organic changes on a valve, such as calcification, fibrosis, and so on. In case of leaflet rupture, the rupture may be closed by an Amplatzer device, which is a soft metal mesh device.

It is noted that in case of an unsuccessful procedure a patient may be operated on with conventional surgical repair.

Reference is now additionally made to FIGS. 23A-23E, which are simplified drawings of example embodiments of the present invention, designed for leaflet stretching working on one side of a leaflet, penetrating through the leaflet, and working on two sides of the leaflet.

FIG. 23A corresponds to FIG. 6F, depicting the third leaflet stretching device 605 of FIG. 6F inside a catheter 620. The third leaflet stretching device 605 is designed to engage a leaflet on one side, and exert a stretching force on the leaflet.

FIG. 23B corresponds to FIG. 14A, depicting the eighth leaflet stretching device 1205 of FIG. 14A after engaging an anterior leaflet 38 with prongs 1225 and blades 1220. A stem 1210 of the eighth leaflet stretching device 1205 penetrates through the anterior leaflet 38, such that the eighth leaflet stretching device 1205 is mostly on one side of the leaflet yet at least the stem 1210 penetrates through the leaflet.

FIG. 23C corresponds to FIG. 16, depicting the eighth leaflet stretching device 1205 of FIG. 16 after engaging an anterior leaflet 38. A stem 1210 of the eighth leaflet stretching device 1205 penetrates through the anterior leaflet 38, such that the eighth leaflet stretching device 1205 is mostly on one side of the leaflet yet at least the stem 1210 penetrates through the leaflet. An optional mesh 1605 slides along the stem 1210 and closes upon the anterior valve leaflet 38 from an opposite side of the leaflet as the rest of the eighth leaflet stretching device 1205.

FIG. 23D corresponds to FIG. 17C, in which the ninth leaflet stretching device of FIG. 17C includes an umbrella shaped portion 1705, having stretching fingers 1715 with prongs 1725 engaging one side of an anterior valve leaflet 38, a stem 1710 penetrating through the anterior valve leaflet 38, and an additional foldable umbrella of blades 1735 having blades 1720 for cutting slits into the anterior valve leaflet 38. The additional foldable umbrella of blades 1735 performs its work on the other side of the anterior valve leaflet 38 from the umbrella shaped portion 1705.

FIG. 23E corresponds to FIG. 17F, in which an umbrella shaped portion 1705, has stretching fingers 1715 with prongs 1725 for engaging one side of a leaflet (not shown) and a stem 1710 for penetrating through the leaflet, and a counter umbrella 1750 has a plurality of spokes 1755 for sliding along the stem 1710. The spokes 1755 perform their work on the opposite side of the leaflet from the umbrella shaped portion 1705, and serve for keeping the leaflet against the umbrella shaped portion 1705, for thereby remodeling the leaflet.

In some of the methods described above the leaflet may be punctured or cut through, producing holes in the leaflet, intentionally and/or unintentionally. In case of small holes, the holes may mend on their own.

In case of larger holes, and especially in case of holes made intentionally, it has been described above that a mesh is inserted to cover the holes and lessen or prevent leakage through the holes.

In some embodiments of the invention the mesh is optionally soft enough to bend with the natural bending of a valve leaflet.

In some embodiments of the invention the mesh optionally consists essentially of a biodegradable material, covering the holes and gradually degrading as the holes heal.

In some embodiments of the invention the mesh optionally consists essentially of a metal mesh.

In some embodiments of the invention the mesh optionally consists essentially of a woven mesh.

It is expected that during the life of a patent maturing from this application many relevant leaflet engaging elements will be developed and the scope of the term leaflet engaging elements is intended to include all such new technologies a priori.

As used herein the term “about” refers to ±20%.

The terms “comprising”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the biological and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for remodeling atrioventricular valve leaflets comprising one or more stretching members and one or more leaflet engaging elements, wherein:
each stretching member comprises at least one leaflet engaging element;
the leaflet engaging elements are configured to penetrate a valve leaflet; and
the stretching members are configured to be positioned externally to a plane of the leaflet when the leaflet engaging elements have penetrated a valve leaflet.

2. The device of claim 1 in which the stretching members are configured to exert a stretching force on the valve leaflet by pushing on the leaflet engaging elements.

3. The device of claim 1 in which the stretching members are configured to exert a stretching force on the valve leaflet by pulling on the leaflet engaging elements.

4-5. (canceled)

6. The device of claim 1 in which the leaflet engaging element comprises a blade.

7. The device of claim 1 in which the leaflet engaging elements are configured to engage the valve leaflet substantially in a shape which is one of a group including:
a circle;
an oval;
a closed curve;
two parallel lines; and
a plurality of concentric circles.

8-9. (canceled)

10. The device of claim 1 in which the leaflet engaging elements are configured to penetrate the leaflet over an area comprising about 20% to 80% of an area of the leaflet.

11-17. (canceled)

18. The device of claim 1 and further comprising a mesh comprising a shape configured for covering at least holes made by the leaflet engaging elements.

19. The device of claim 1 and further comprising a counter plate, the counter plate comprising blades for cutting into the valve leaflet, the blades configured to cut into the valve leaflet within an area encompassed by the stretching members.

20. (canceled)

21. The device of claim 19, in which at least one of the one or more stretching members is connected to a stem, and the counter plate further comprises a hole for sliding over the stem.

22. (canceled)

23. A method for remodeling atrioventricular valve leaflets comprising:
inserting one or more leaflet engaging elements into an atrioventricular valve leaflet; and
using stretching members positioned externally to a plane of the leaflet to apply a stretching force on the atrioventricular valve leaflet.

24. The method of claim 23 in which the stretching force is applied by forcing apart the leaflet engaging elements.

25. (canceled)

26. The method of claim 23 in which the stretching force is applied by pulling on the leaflet engaging elements.

27. The method of claim 23 in which the stretching force is enough to cause elastic stretching of the leaflet, and less than enough to cause plastic deformation of the leaflet.

28. The method of claim 23 in which the stretching force is enough to cause plastic deformation of the leaflet.

29. The method of claim 23 in which the leaflet engaging elements are left in the atrioventricular valve leaflet and the forcing continues over a period of time.

30-31. (canceled)

32. The method of claim 23 and further comprising using stretching fingers attached to the leaflet engaging elements for forcing apart the leaflet engaging elements.

33. (canceled)

34. The method of claim 23 and further comprising cutting holes into the atrioventricular valve leaflet.

35. The method of claim 34 and further comprising covering holes made by the cutting with a mesh.

36. The method of claim 23 and further comprising bringing the one or more leaflet engaging elements up to an in-vivo atrioventricular valve leaflet via a catheter.

37. (canceled)

38. A device for remodeling atrioventricular valve leaflets comprising:
one or more means for grasping one or more locations on an atrioventricular valve leaflet; and
means, positioned externally to a plane of the leaflet, for applying a stretching force between the locations.

39. The apparatus of claim 38 and further including means for cutting holes into the atrioventricular valve leaflet.

40. The apparatus of claim 39 and further including means for covering up the holes in the atrioventricular valve leaflet.
